(19) United States
(12) Patent Application Publication
(10) Pub. No.: US 2026/0047995 A1
(43) Pub. Date: Feb. 19, 2026

(54) COSMETIC COMPOSITION OF STABILIZED RETINOID BY PH BUFFER

(71) Applicant: MARY KAY INC., Addison, TX (US)

(72) Inventors: Lee VICKERS, Addison, TX (US); Phi DOAN, Addison, TX (US); Habte YAYEHYIRAD, Addison, TX (US); Yuan TANG, Addison, TX (US); Tiffany CARLE, Addison, TX (US); Wanli ZHAO, Addison, TX (US); An HUYNH, Addison, TX (US)

(21) Appl. No.: 19/299,567

(22) Filed: Aug. 14, 2025

Related U.S. Application Data
(60) Provisional application No. 63/683,092, filed on Aug. 14, 2024.
(60) Provisional application No. 63/778,971, filed on Mar. 27, 2025.

(51) Int. Cl. A61K 8/67; A61K 8/06; A61Q 19/00

(52) U.S. Cl. CPC A61K 8/671; A61K 8/062; A61K 8/678; A61Q 19/00; A61K 2800/522

(57) ABSTRACT

Retinoids in cosmetic compositions can degrade over time, thereby diminishing the effectiveness of cosmetic compositions. The present disclosure includes cosmetic compositions that exhibit enhanced retinoid stability. Also disclosed are methods of improving the stability of retinoids in cosmetic compositions. Additives can be used, either alone or in combination, to improve retinoid stability and increase retinoid-containing cosmetic composition shelf life.

### CROSS-REFERENCE TO RELATED APPLICATIONS

This application claims the benefit of priority to U.S. Provisional Patent Applications Ser. No. 63/683,092, filed Aug. 14, 2024, and 63/778,971, filed Mar. 27, 2025, which are hereby incorporated by reference in their entireties.

### BACKGROUND

### A. Technical Field

The present disclosure relates to methods for improving the stability of retinoids in cosmetic compositions, and cosmetic compositions stabilized by the method.

### B. Description of Related Art

Retinoids are dermatological agents that are effective against acne, psoriasis, skin aging, and other skin conditions. However, their susceptibility to degradation is a limiting factor for their widespread use in skincare products. Retinoids are thermolabile, photosensitive, and susceptible to oxidation, and can be degraded to inactive compounds when exposed to increased temperatures (including room temperature), light, and/or air. For example, the retinoid retinol can be oxidized to retinaldehyde when exposed to light and air, which can then be oxidized to retinoic acid. Retinoic acid can then be oxidized to inactive hydroxylated products like 4-hydroxyl- and 18-hydroxyl retinoic acid.

Typical skincare products are packaged in containers that are stored at room temperature. During each use, most containers are opened and the product is exposed to air. Repeated exposure to the oxygen in air can lead to oxidative degradation and reduced effectiveness of retinoid-containing products. In addition to storage conditions (temperature, light, and oxygen in air), packaging container materials, as well as other ingredients in skincare products, can influence the degradation rate of retinoids.

The effectiveness of retinoids is significantly reduced when retinoids are degraded by oxidation. Maintaining retinoid stability in skincare products remains a problem for both cosmeticproducers and consumers. Thus, there remains a need for methods of preventing of improving retinoid stability and reducing undesired retinoid degradation in skincare products.

### SUMMARY

The inventors have identified a solution to the problems associated with unacceptable degradation of retinoids in cosmetic products. The solution resides in the use of one or more active agents that imparts stability to retinoids in cosmetic products. The one or more active agents can help stabilize retinoids and reduce retinoid degradation rates. The use of one or more active agents is especially useful for cosmetic products that are stored for extended durations or at increased temperatures, and/or repeatedly exposed to oxygen in air.

An object of the present invention is to provide a method for increasing stability of retinoids. In one instance, there is disclosed a method of improving the stability of a retinoid in a retinoid-containing composition, the method comprising adding a buffer to the retinoid-containing composition in an amount that is sufficient to maintain a pH of the composition in a pH range of from 5 to 8. In some embodiments, the buffer is added to the retinoid-containing composition in an amount that is sufficient to maintain a pH of the composition at a pH that is any one of, less than, greater than, between, or any range thereof of 5.0, 5.1, 5.2, 5.3, 5.4, 5.5, 5.6, 5.7, 5.8, 5.9, 6.0, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, and 8.0. In some embodiments, the cosmetic composition is an oil-in-water emulsion. In some embodiments, the method increases retinoid stability such that at least 90% by weight of an original amount of the retinoid remains after storage for 12 weeks at 40° C.

In some embodiments, the retinoid is selected from retinol, retinal, retinoic acid, branched or unbranched C1-C20 esters of retinoic acid, etretinate, acitretin, adapalene, bexarotene, tazarotene, and any combination thereof. In some embodiments, the retinoid is present in an amount ranging from 0.01 wt. % to 5 wt. % relative to a weight of the composition. In further embodiments, the retinoid is present in an amount ranging from 1 wt. % to 3 wt. % relative to a weight of the composition. Thus, it is contemplated that the retinoid is present in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein. In some embodiments, the buffer is selected from the group consisting of tris(hydroxymethyl)aminomethane (TRIS), citrate phosphate, 2-(N-morpholino)ethanesulfonic acid (MES), 3-(N-morpholino)propanesulfonic acid (MOPS), 4-(N-morpholino)butanesulfonic acid (MOBS), bis(2-hydroxyethyl) amino-tris(hydroxymethyl)methane (BIS-TRIS), N-(2-acetamido) iminodiacetic acid (ADA), N-(2-acetamido)-2-aminoethanesulfonic acid (ACES), 1,4-piperazinediethanesulfonic acid (PIPES), 3-morpholino-2-hydroxypropane sulfonic acid (MOPSO), 1,3-bis[tris(hydroxymethyl)methylamino]propane (BIS-TRIS propane), N,N-bis-(2-hydroxyethyl)-2-aminoethanesulfonic acid (BES), 2-[(2-hydroxy-1,1-bis-(hydroxymethyl)ethyl)amino] ethanesulfonic acid (TES), 4-(2-hydroxyethyl) piperazine-1-ethanesulfonic acid (HEPES), 2-hydroxy-3-[tris(hydroxymethyl)methylamino]-1-propanesulfonic acid (TAPSO), 2-amino-2-(hydroxymethyl)-1,3-propanediol (trizma), piperazine-N,N′-bis(2-hydroxypropanesulfonic acid) (POPSO), 4-(2-hydroxyethyl)-1-piperazinepropanesulfonic acid (EPPS), Diglycine, N,N-bis(2-hydroxyethyl)glycine (BICINE), N-(2-hydroxyethyl)piperazine-N′-(4-butanesulfonic acid) (HEPBS), 2-amino-2-methyl-1,3-propanediol, and N-[tris(hydroxymethyl)methyl]-3-aminopropanesulfonic acid (TAPS). In some embodiments, the buffer is added in an amount ranging from 0.01 wt. % to 5 wt. % relative to the weight of the composition. Thus, it is contemplated that the buffer is added in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein.

In some embodiments, a method of improving the stability of a retinoid further comprises adding at least one antioxidant to the retinoid-containing composition. In some embodiments, the at least one water-soluble antioxidant is selected from the group consisting of citric acid, a citrate salt, ascorbic acid, an ascorbate salt, and a combination thereof. In some embodiments, the at least one oil-soluble antioxidant is selected from the group consisting of tocopherol, a tocopherol ester, and a combination thereof. In some embodiments, the at least one water-soluble antioxidant is added in an amount ranging from 0.01 wt. % to 5 wt. % relative to the weight of the composition. Thus, it is contemplated that the at least one water-soluble antioxidant is added in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein. In some embodiments, the at least one oil-soluble antioxidant is added in an amount ranging from 0.01 wt. % to 5 wt. % relative to the weight of the composition. Thus, it is contemplated that the at least oil-soluble antioxidant is added in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein.

In some embodiments, a method of improving the stability of a retinoid further comprises adding at least one chelating agent to the retinoid-containing composition. In some embodiments, the at least one chelating agent is selected from disodium EDTA, tetrasodium EDTA, citric acid, glucono δ-lactone, sodium phytate, and any combination thereof. In some embodiments, the at least one chelating agent is added in an amount ranging from 0.01 wt. % to 5 wt. % relative to the weight of the composition. Thus, it is contemplated that the at least one chelating agent is added in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein.

In another aspect, disclosed is a cosmetic composition comprising at least one retinoid and a buffer, wherein the buffer is added in an amount that is sufficient to maintain a pH of the cosmetic composition in a pH range of from 5 to 8, and wherein the cosmetic composition is in the form of an oil-in-water emulsion. In one instance the retinoid-containing composition above includes any one of, any combination of, or all of a buffer, at least one antioxidant, at least one chelating agent, an emulsifier, an emulsion thickening agent, an emulsion stabilizer, a surfactant, a texture enhancer, an emollient, a humectant, a preservative, a pH-adjusting agent, and a cleansing agent. The amounts of the ingredients within the composition can vary (e.g., amounts can be aslow as 0.000001% to as high as 80% w/w or any range therein). The composition may further comprise one or more ingredients described herein. For example, the composition may comprise one or more additional ingredients selected from one or more conditioning agents, moisturizing agents, pH adjusters, structuring agents, inorganic salts, and preservatives.

In some embodiments, the at least one retinoid is selected from retinol, retinal, retinoic acid, and esters of retinoic acid, etretinate, acitretin, adapalene, bexarotene, tazarotene, and any combination thereof. In some embodiments, the at least one retinoid is present in an amount ranging from 0.01 wt. % to 5 wt. % relative to the weight of the composition. In further embodiments, the at least one retinoid is present in an amount ranging from 1 wt. % to 3 wt. % relative to a weight of the composition. Thus, it is contemplated that the at least one retinoid is present in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein. In some embodiments, the buffer is selected from the group consisting of tris(hydroxymethyl)aminomethane (TRIS), citrate phosphate, 2-(N-morpholino)ethanesulfonic acid (MES), 3-(N-morpholino)propanesulfonic acid (MOPS), 4-(N-morpholino)butanesulfonic acid (MOBS), bis(2-hydroxyethyl)amino-tris(hydroxymethyl)methane (BIS-TRIS), N-(2-acetamido) iminodiacetic acid (ADA), N-(2-acetamido)-2-aminoethanesulfonic acid (ACES), 1,4-piperazinediethanesulfonic acid (PIPES), 3-morpholino-2-hydroxypropane sulfonic acid (MOPSO), 1,3-bis[tris(hydroxymethyl)methylamino]propane (BIS-TRIS propane), N,N-bis-(2-hydroxyethyl)-2-aminoethanesulfonic acid (BES), 2-[(2-hydroxy-1,1-bis-(hydroxymethyl)ethyl)amino]ethanesulfonic acid (TES), 4-(2-hydroxyethyl) piperazine-1-ethanesulfonic acid (HEPES), 2-hydroxy-3-[tris(hydroxymethyl)methylamino]-1-propanesulfonic acid (TAPSO), 2-amino-2-(hydroxymethyl)-1,3-propanediol (trizma), piperazine-N,N′-bis(2-hydroxypropanesulfonic acid) (POPSO), 4-(2-hydroxyethyl)-1-piperazinepropanesulfonic acid (EPPS), Diglycine, N,N-bis(2-hydroxyethyl)glycine (BICINE), N-(2-hydroxyethyl)piperazine-N′-(4-butanesulfonic acid) (HEPBS), 2-amino-2-methyl-1,3-propanediol, and N-[tris(hydroxymethyl)methyl]-3-aminopropanesulfonic acid (TAPS). In some embodiments, the buffer is added in an amount ranging from 0.01 wt. % to 5 wt. % relative to the weight of thecomposition. Thus, it is contemplated that the buffer is added in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein.

In some embodiments, the cosmetic composition further comprises at least one antioxidant. In some embodiments, the at least one antioxidant is at least one water-soluble antioxidant, at least one oil-soluble antioxidant, or a combination thereof. In some embodiments, the at least one water-soluble antioxidant is selected from the group consisting of citric acid, a citrate salt, ascorbic acid, an ascorbate salt, and a combination thereof. In some embodiments, the oil-soluble antioxidant is selected from the group consisting of tocopherol, a tocopherol ester, and a combination thereof. In some embodiments, the water-soluble antioxidant is added in an amount ranging from 0.01 wt. % to 5 wt. % relative to the weight of the composition. Thus, it is contemplated that the water-soluble antioxidant is added in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein. In some embodiments, the oil-soluble antioxidant is added in an amount ranging from 0.01 wt. % to 5 wt. % relative to the weight of the composition. Thus, it is contemplated that the oil-soluble antioxidant is added in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein. In some embodiments, the cosmetic composition further comprises at least one chelating agent. In some embodiments, the at least one chelating agent is selected from the group consisting of disodium EDTA, tetrasodium EDTA, citric acid, glucono 8-lactone, sodium phytate, and any combination thereof. In some embodiments, the at least one chelating agent is added in an amount ranging from 0.01 wt. % to 5 wt. % relative to the weight of the composition. Thus, it is contemplated that the at least one chelating agent is added in an amount that is any one of, less than, greater than, or between 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. % relative to the weight of the composition, or any range derivable therein. In some embodiments, the composition exhibits enhanced retinoid stability such that at least 90% by weight of an original amount of the retinoid in the cosmetic composition remains after storage for 12 weeks at 40° C. In some embodiments, the composition further comprises further comprising at least one of an emulsifier, an emulsion thickening agent, an emulsion stabilizer, a surfactant, a texture enhancer, an emollient, a humectant, a preservative, a pH-adjusting agent, and a cleansing agent.

In another aspect, disclosed is a method for extending the effectiveness of a retinoid-containing composition, comprising adding a buffer to the retinoid-containing composition in an amount that is sufficient to maintain a pH of the composition in a pH range of from 5 to 8, wherein the wherein the cosmetic composition is an oil-in-water emulsion. Methods of use for the compositions disclosed herein are also disclosed. In some instances, the methods comprise topically applying any one of the compositions disclosed herein to skin and/or the face and/or eye area in need thereof. In one aspect, any one of the compositions disclosed herein are topically applied and the composition is left on the application area, removed from the application area after a period of time, and/or removed directly after application.

In particular aspects, the compositions of the present invention are formulated as a topical skin composition. The composition can have a dermatologically acceptable vehicle or carrier for the compounds, compositions and extracts. The composition can further include a moisturizing agent or a humectant, a surfactant, a silicone containing compounds, a UV agent, an oil, and/or other ingredients identified in this specification or those known in the art. The composition can be a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solutions (e.g., aqueous or hydro-alcoholic solutions), anhydrous bases (e.g., lipstick or a powder), ointments, milk, paste, aerosol, solid forms, eye jellies, etc. The composition can be in powdered form (e.g., dried, lyophilized, particulate, etc.). The composition can be formulated for topical skin application at least 1, 2, 3, 4, 5, 6, 7, or more times a day during use. In some aspects of the present invention, compositions can be storagestable or color stable, or both. It is also contemplated that the viscosity of the composition can be selected to achieve a desired result, e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps, etc., as measured on a Brookfield Viscometer using a TC spindle at 2.5 rpm at 25° C.).

The compositions of the present invention can also be modified to have a desired oxygen radical absorbance capacity (ORAC) value. In certain non-limiting aspects, the compositions of the present invention or the component or extracts thereof identified throughout this specification can be modified to have an ORAC value per mg of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 95, 100, 200, 300, 400, 500, 600, 700, 800, 900, 1000, 2000, 3000, 4000, 5000, 6000, 7000, 8000, 9000, 10000, 15000, 20000, 30000, 50000, 100000 or more or any range derivable therein.

The compositions, in non-limiting aspects, can have a pH of about 5 to about 8. In some aspects, the pH can be 5.0, 5.1, 5.2, 5.3, 5.4, 5.5, 5.6, 5.7, 5.8, 5.9, 6.0, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. The compositions can include a triglyceride. Non-limiting examples include small, medium, and large chain triglycerides. In certain aspects, the triglyceride is a medium chain triglyceride (e.g., caprylic capric triglyceride). The compositions can also include preservatives. Non-limiting examples of preservatives include phenoxyethanol, methylparaben, propylparaben, or any mixture of thereof. In some embodiments, the composition is paraben-free.

Compositions of the present invention can have UVA and UVB absorption properties. The compositions can have an sun protection factor (SPF) of 2, 3, 4, 5, 6, 7, 8, 9, 10, 11, 12, 13, 14, 15, 20, 25, 30, 35, 40, 45, 50, 55, 60, or more, or any integer or derivative therein. The compositions can be sunscreen lotions, sprays, or creams.

The compositions of the present invention can also include any one of, any combination of, or all of the following additional ingredients: water, a conditioning agent, a chelating agent, a moisturizing agent, a pH adjuster, inorganic salts, a preservative, a thickening agent, a silicone containing compound, an essential oil, a structuring agent, a vitamin, a pharmaceutical ingredient, or an antioxidant, or any combination of such ingredients or mixtures of such ingredients. In certain aspects, the composition can include at least two, three, four, five, six, seven, eight, nine, ten, or more, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

Kits that include the compositions of the present invention are also contemplated. In certain embodiments, the composition is comprised in a container. The container can be a bottle, dispenser, or package. The container can dispense a pre-determined amount of the composition. In certain aspects, the compositions is dispensed in a spray, mist, dollop, or liquid. The container can include indicia on its surface. The indicia can be a word, an abbreviation, a picture, or a symbol.

It is also contemplated that the compositions disclosed throughout this specification can be used as a leave-on or rinse-off composition. By way of example, a leave-on composition can be one that is topically applied to skin and remains on the skin for a period of time (e.g., at least 5, 6, 7, 8, 9, 10, 20, or 30 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). Alternatively, a rinse-off composition can be a product that is intended to be applied to the skin and then removed or rinsed from the skin (e.g., with water) within a period of time such as less than 5, 4, 3, 2, or 1 minute. An example of a rinse off composition can be a skin cleanser, shampoo, conditioner, or soap. An example of a leave-on composition can be a skin moisturizer, sunscreen, mask, overnight cream, or a day cream.

It is contemplated that any embodiment discussed in this specification can be implemented with respect to any method or composition of the invention, and vice versa. Furthermore, compositions of the invention can be used to achieve methods of the invention.

In some embodiments, compositions of the present invention can be pharmaceutically or cosmetically elegant or can have pleasant tactile properties. “Pharmaceutically elegant,” “cosmetically elegant,” and/or “pleasant tactile properties” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

Also contemplated is a product comprising a composition of the present invention. In non-limiting aspects, the product can be a cosmetic product. The cosmetic product can be those described in other sections of this specification or those known to a person of skill in the art. Non-limiting examples of products include a moisturizer, a cream, a lotion, a skin softener, a serum, a gel, a wash, a body butter, a scrub, a foundation, a night cream, a lipstick, a cleanser, a toner, a sunscreen, a mask, an anti-aging product, a deodorant, an antiperspirant, a perfume, a cologne, etc.

Also disclosed in the context of the present invention are aspects 1 to 34. Aspect 1 is method of improving the stability of a retinoid in a retinoid-containing composition, the method comprising adding a buffer to the retinoid-containing composition in an amount that is sufficient to maintain a pH of the composition in a pH range of from 5 to 8, wherein the cosmetic composition is an oil-in-water emulsion. Aspect 2 depends on Aspect 1, wherein the retinoid is selected from retinol, retinal, retinoic acid, branched or unbranched C1-C20 esters of retinoic acid, etretinate, acitretin, adapalene, bexarotene, tazarotene, and any combination thereof. Aspect 3 depends on Aspect 1, wherein the retinoid is present in an amount ranging from 0.01 wt. % to 5 wt. % relative to a weight of the composition. Aspect 4 depends on Aspect 1, wherein the retinoid is present in an amount ranging from 0.01 wt. % to 5 wt. % relative to a weight of the composition. Aspect 5 depends on Aspect 1, wherein the buffer is selected from the group consisting of tris(hydroxymethyl)aminomethane (TRIS), citrate phosphate, 2-(N-morpholino)ethanesulfonic acid (MES), 3-(N-morpholino)propanesulfonic acid (MOPS), 4-(N-morpholino)butanesulfonic acid (MOBS), bis(2-hydroxyethyl)amino-tris(hydroxymethyl)methane (BIS-TRIS), N-(2-acetamido) iminodiacetic acid (ADA), N-(2-acetamido)-2-aminoethanesulfonic acid (ACES), 1,4-piperazinediethanesulfonic acid (PIPES), 3-morpholino-2-hydroxypropane sulfonic acid (MOPSO), 1,3-bis[tris(hydroxymethyl)methylamino]propane (BIS-TRIS propane), N,N-bis-(2-hydroxyethyl)-2-aminoethanesulfonic acid (BES), 2-[(2-hydroxy-1,1-bis-(hydroxymethyl)ethyl)amino]ethanesulfonic acid (TES), 4-(2-hydroxyethyl) piperazine-1-ethanesulfonic acid (HEPES), 2-hydroxy-3-[tris(hydroxymethyl)methylamino]-1-propanesulfonic acid (TAPSO), 2-amino-2-(hydroxymethyl)-1,3-propanediol (trizma), piperazine-N,N′-bis(2-hydroxypropanesulfonic acid) (POPSO), 4-(2-hydroxyethyl)-1-piperazinepropanesulfonic acid (EPPS), Diglycine, N,N-bis(2-hydroxyethyl)glycine (BICINE), N-(2-hydroxyethyl)piperazine-N′-(4-butanesulfonic acid) (HEPBS), 2-amino-2-methyl-1,3-propanediol, and N-[tris(hydroxymethyl)methyl]-3-aminopropanesulfonic acid (TAPS). Aspect 6 depends on Aspect 1, wherein the buffer is added in an amount ranging from 0.01 wt. % to 5 wt. % relative to the weight of the composition. Aspect 7 depends on Aspect 1, further comprising adding at least one antioxidant to the retinoid-containing composition. Aspect 8 depends on Aspect 7, wherein the at least one antioxidant is at least one water-soluble antioxidant, at least one oil-soluble antioxidant, or a combination thereof. Aspect 9 depends on Aspect 8, wherein the at least one water-soluble antioxidant is selected from the group consisting of citric acid, a citrate salt, ascorbic acid, an ascorbate salt, and a combination thereof. Aspect 10 depends on Aspect 8, wherein the at least one oil-soluble antioxidant is selected from the group consisting of tocopherol, a tocopherol ester, and a combination thereof. Aspect 11 depends on Aspect 9, wherein the at least one water-soluble antioxidant is in an amount ranging from 0.01 wt. % to 5 wt. % relative to the weight of the composition. Aspect 12 depends on Aspect 10, wherein the at least one oil-soluble antioxidant is in an amount ranging from 0.01 wt. % to 5 wt. % relative to the weight of the composition. Aspect 13 depends on Aspect 1, further comprising adding at least one chelating agent to the retinoid-containing composition. Aspect 14 depends on Aspect 13, wherein the at least one chelating agent is selected from disodium EDTA, tetrasodium EDTA, citric acid, glucono δ-lactone, sodium phytate, and any combination thereof. Aspect 15 depends on Aspect 14, wherein the at least one chelating agent is in an amount ranging from 0.01 wt. % to 5 wt. % relative to the weight of the composition. Aspect 16 depends on Aspect 1, wherein the method increases retinoid stability such that at least 90% by weight of an original amount of the retinoid remains after storage for 12 weeks at 40° C. Aspect 17 is a cosmetic composition comprising at least one retinoid and a buffer, wherein the buffer is present in an amount that is sufficient to maintain a pH of the composition in a pH range of from 5 to 8, and wherein the cosmetic composition is in the form of an oil-in-water emulsion. Aspect 18 depends on Aspect 17 wherein the at least one retinoid is selected from retinol, retinal, retinoic acid, and esters of retinoic acid, etretinate, acitretin, adapalene, bexarotene, tazarotene, and any combination thereof. Aspect 19 depends on Aspect 17, wherein the at least one retinoid is present in an amount ranging from 0.01 wt. % to 5 wt. % relative to the weight of the composition. Aspect 20 depends on Aspect 17, wherein the at least one retinoid is present in an amount ranging from 0.01 wt. % to 5 wt. % relative to a weight of the composition. Aspect 21 depends on Aspect 17 wherein the buffer is selected from the group consisting of tris(hydroxymethyl)aminomethane (TRIS), citrate phosphate, 2-(N-morpholino)ethanesulfonic acid (MES), 3-(N-morpholino)propanesulfonic acid (MOPS), 4-(N-morpholino)butanesulfonic acid (MOBS), bis(2-hydroxyethyl)amino-tris(hydroxymethyl)methane (BIS-TRIS), N-(2-acetamido) iminodiacetic acid (ADA), N-(2-acetamido)-2-aminoethanesulfonic acid (ACES), 1,4-piperazinediethanesulfonic acid (PIPES), 3-morpholino-2-hydroxypropane sulfonic acid (MOPSO), 1,3-bis[tris(hydroxymethyl)methylamino]propane (BIS-TRIS propane), N,N-bis-(2-hydroxyethyl)-2-aminoethanesulfonic acid (BES), 2-[(2-hydroxy-1,1-bis-(hydroxymethyl)ethyl)amino]ethanesulfonic acid (TES), 4-(2-hydroxyethyl) piperazine-1-ethanesulfonic acid (HEPES), 2-hydroxy-3-[tris(hydroxymethyl)methylamino]-1-propanesulfonic acid (TAPSO), 2-amino-2-(hydroxymethyl)-1,3-propanediol (trizma), piperazine-N,N′-bis(2-hydroxypropanesulfonic acid) (POPSO), 4-(2-hydroxyethyl)-1-piperazinepropanesulfonic acid (EPPS), Diglycine, N,N-bis(2-hydroxyethyl)glycine (BICINE), N-(2-hydroxyethyl)piperazine-N′-(4-butanesulfonic acid) (HEPBS), 2-amino-2-methyl-1,3-propanediol, and N-[tris(hydroxymethyl)methyl]-3-aminopropanesulfonic acid (TAPS). Aspect 22 depends on Aspect 17, wherein the buffer is present in an amount ranging from 0.01 wt. % to 5 wt. % relative to the weight of the composition. Aspect 23 depends on Aspect 17, further comprising at least one antioxidant. Aspect 24 depends on Aspect 23, wherein the at least one antioxidant is at least one water-soluble antioxidant, at least one oil-soluble antioxidant, or a combination thereof. Aspect 25 depends on Aspect 24, wherein the at least one water-soluble antioxidant is selected from the group consisting of citric acid, a citrate salt, ascorbic acid, an ascorbate salt, and a combination thereof. Aspect 26 depends on Aspect 24, wherein the oil-soluble antioxidant is selected from the group consisting of tocopherol, a tocopherol ester, and a combination thereof. Aspect 27 depends on Aspect 24, wherein the water-soluble antioxidant is present in an amount ranging from 0.01 wt. % to 5 wt. % relative to the weight of the composition. Aspect 28 depends on Aspect 24, wherein the oil-soluble antioxidant is present in an amount ranging from 0.01 wt. % to 5 wt. % relative to the weight of the composition. Aspect 29 depends on Aspect 17, further comprising at least one chelating agent. Aspect 30 depends on Aspect 29, wherein the at least one chelating agent is selected from the group consisting of disodium EDTA, tetrasodium EDTA, citric acid, glucono δ-lactone, sodium phytate, and any combination thereof. Aspect 31 depends on Aspect 30, wherein the at least one chelating agent is present in an amount ranging from 0.01 wt. % to 5 wt. % relative to the weight of the composition. Aspect 32 depends on Aspect 17, wherein the composition exhibits enhanced retinoid stability such that at least 90% by weight of an original amount of the retinoid in the cosmetic composition remains after storage for 12 weeks at 40° C. Aspect 33 depends on Aspect 17, further comprising at least one of an emulsifier, an emulsion thickening agent, an emulsion stabilizer, a surfactant, a texture enhancer, an emollient, a humectant, a preservative, a pH-adjusting agent, and a cleansing agent. Aspect 34 depends on Aspect 17, wherein the composition comprises 5.0 to 95.0% by weight of water; 1.0 to 10.0% by weight of isononyl isononanoate; 1.0 to 10.0% by weight of methyl trimethicone; 1.0 to 10.0% by weight of neopentyl glycol diheptanoate; 1.0 to 10.0% by weight of glycerin; 1.0 to 10.0% by weight of silica; 0.1 to 5.0% by weight of ammonium acryloyldimethyltaurate/VP copolymer; 0.1 to 5.0% by weight of glyceryl stearate; 0.1 to 5.0% by weight of PEG-100 stearate; 0.1 to 5.0% by weight of hydroxyethyl urea; 0.1 to 5.0% by weight of ceteareth-33; 0.1 to 5.0% by weight of tocopheryl acetate; 0.1 to 5.0% by weight of phenoxyethanol; 0.1 to 5.0% by weight of polysorbate-20; 0.1 to 5.0% by weight of caprylyl glycol; 0.1 to 0.5% by weight of disodium phosphate; 0.1 to 0.5% by weight of retinol; 0.1 to 0.5% by weight of hydroxyacetophenone; 0.1 to 0.5% by weight of citric acid; 0.1 to 0.5% by weight of disodium EDTA; 0.1 to 0.5% by weight of potassium hydroxide; 0.1 to 0.5% by weight of xanthan gum; 0.01 to 0.5% by weight of ascorbic acid; 0.01 to 0.2% by weight of silica silylate; and 0.01 to 0.1% by weight of ammonium lactate. Aspect 35 depends on Aspect 17, wherein the composition comprises about 66.4% by weight of water; about 5.0% by weight of isononyl isononanoate; about 5.0% by weight of methyl trimethicone; about 5.0% by weight of neopentyl glycol diheptanoate; about 5.0% by weight of glycerin; about 3.5% by weight of silica; about 1.5% by weight of ammonium acryloyldimethyltaurate/VP copolymer; about 1.4% by weight of glyceryl stearate; about 1.3% by weight of PEG-100 stearate; about 1.2% by weight of hydroxyethyl urea; about 1.0% by weight of ceteareth-33; about 0.5% by weight of tocopheryl acetate; about 0.5% by weight of phenoxyethanol; about 0.4% by weight of polysorbate-20; about 0.1% by weight of caprylyl glycol; about 0.3% by weight of disodium phosphate; about 0.3% by weight of retinol; about 0.25% by weight of hydroxyacetophenone; about 0.2% by weight of citric acid; about 0.2% by weight of disodium EDTA; about 0.16% by weight of potassium hydroxide; about 0.15% by weight of xanthan gum; about 0.1% by weight of ascorbic acid; about 0.1% by weight of silica silylate; and about 0.04% by weight of ammonium lactate. Aspect 36 is a method for extending the effectiveness of a retinoid-containing composition, comprising adding a buffer to the retinoid-containing composition in an amount that is sufficient to maintain a pH of the composition in a pH range of from 5 to 8, wherein the wherein the cosmetic composition is an oil-in-water emulsion.

“Topical application” means to apply or spread a composition onto the surface of lips or keratinous tissue. “Topical skin composition” includes compositions suitable for topical application on skin, lips, and/or keratinous tissue. Such compositions are typically dermatologically-acceptable in that they do not have undue toxicity, incompatibility, instability, allergic response, and the like, when applied to lips, skin, and/or keratinous tissue. Topical skin care compositions of the present invention can have a selected viscosity to avoid significant dripping or pooling after application to lips, skin, and/or keratinous tissue.

“Keratinous tissue” includes keratin-containing layers disposed as the outermost protective covering of mammals and includes, but is not limited to, lips, skin, hair and nails.

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or any variation of these terms includes any measurable decrease or complete inhibition to achieve a desired result. The terms “promote” or “increase” or any variation of these terms includes any measurable increase, such as a measurable increase of a protein or molecule (e.g., matrix proteins such as fibronectin, laminin, collagen, or elastin or molecules such as hyaluronic acid) to achieve a desired result.

The term “effective,” as that term is used in the specification and/or claims, means adequate to accomplish a desired, expected, or intended result.

The use of the word “a” or “an” when used in conjunction with the terms “comprising,” “including,” “having,” or “containing,” or any variations of these terms, in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the phrase “consisting essentially of,” a basic and novel property of the compositions and methods of the present invention is the ability to improve the stability of a retinoid in a retinoid-containing composition.

The phrase “and/or” means “and” or “or”. To illustrate, A, B, and/or C includes: A alone, B alone, C alone, a combination of A and B, a combination of A and C, a combination of B and C, or a combination of A, B, and C.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings illustrate by way of example and not limitation.

FIG. 1 includes data from HPLC assays of accelerated-aging studies of a retinol-containing cream that included a citrate-phosphate buffer pH=7 stored at 25° C.

FIG. 2 includes data from HPLC assays of accelerated-aging studies of a retinol-containing cream that included a citrate-phosphate buffer pH=7 stored at 40° C.

FIG. 3 includes data from HPLC assays of accelerated-aging studies of a retinol-containing cream that included a citrate-phosphate buffer pH=6 stored at 25° C.

FIG. 4 includes data from HPLC assays of accelerated-aging studies of a retinol-containing cream that included a citrate-phosphate buffer pH=6 stored at 40° C.

FIG. 5 includes data from HPLC assays of accelerated-aging studies of a retinol-containing cream that included a TRIS buffer pH=7 stored at 25° C.

FIG. 6 includes data from HPLC assays of accelerated-aging studies of a retinol-containing cream that included a TRIS buffer pH=7 stored at 40° C.

FIGS. 7A-7B. FIG. 7A is a table with HPLC assay results of a retinol-containing creams stored at different temperatures. The retinol-containing creams included a citrate-phosphate buffer pH=6.5. FIG. 7B is a table of total remaining retinol concentrations of retinol-containing samples stored at different temperatures. The retinol-containing samples included a citrate-phosphate buffer pH=6.5.

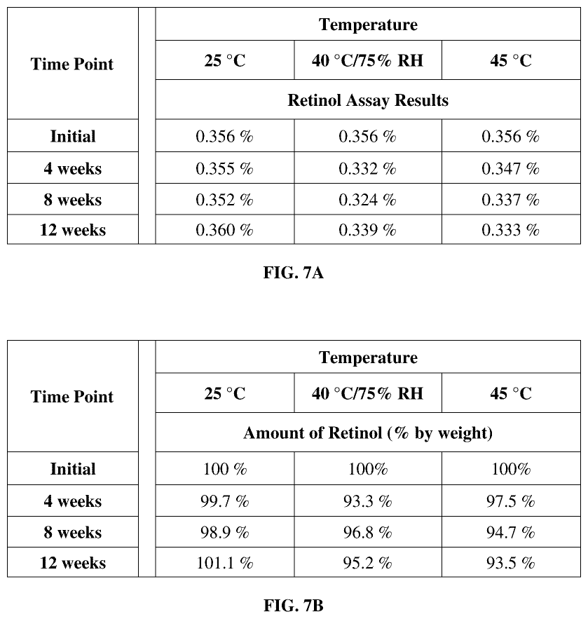

### DETAILED DESCRIPTION

As noted above, the present inventors discovered that retinoids can be protected against chemical and physical degradation by stabilizing the pH of personal care compositions such that the pH is maintained in a range of from 5 to 8. The stability of a retinoid can be measured as the percentage of an active retinoid compound in a composition found in its original form after storage of the composition for a given period of time at a given temperature.

Changes to a composition's pH can be caused by a variety of factors, including component auto-oxidation resulting from absorption of atmospheric oxygen, light-induced degradation, production of carbonic acid resulting from absorption of atmospheric carbon dioxide, leaching of container materials, and introduction of exogenic components resulting from a user's repeated use. Although some personal care compositions are provided at a pH of from 5 to 8, many are not capable of resisting changes to pH. The present inventors discovered that the deliberate stabilization of pH by employing a buffer that resists changes in pH and maintains pH within the desired range provides a means by which retinoid degradation can be reduced.

This approach can be used to produce retinoid-containing personal care compositions with increased shelf lives. The methods and compositions disclosed herein provide a direct financial benefit to the consumer, as a retinoid-containing personal care composition will remain effective for a longer period of time and reduce the frequency at which the consumer has to replace aged and ineffective product. Some embodiments of the present disclosure are therefore directed to methods of improving the stability of a retinoid in a retinoid-containing composition. Some embodiments of the present disclosure are directed to methods and compositions for reducing deep lines in a subject's skin. In some embodiments, the methods and compositions disclosed herein can be used to reduce fine lines in a subject's skin. In some embodiments, the methods and compositions disclosed herein are used to increase firmness in a subject's skin. In some embodiments, the methods and compositions disclosed herein can reduce mottled hyperpigmentation of a subject's skin. In some embodiments, methods and compositions for improving the radiance of a subject's skin are disclosed. In some embodiments, the methods and compositions disclosed herein are employed to improve the texture and/or smoothness of a subject's skin. In some embodiments, the methods and compositions disclosed herein are useful in reducing uneven skin tone of a subject's skin. In some embodiments, the methods and compositions disclosed herein can be used to reduce wrinkles in a subject's skin.

These and other non-limiting aspects of the present invention are described in the following sections.

### A. Active Ingredients

As explained above, topical skin care compositions of the present invention can include one or more retinoids. Retinoids are a group of compounds with cell-regulating effects. They can induce the biosynthesis of collagen and reduce the expression of MMP 1, a protein involved in skin wrinkling. Retinoids have shown positive effects on extrinsic and intrinsic skin aging and has a strong positive effect on collagen metabolism. The term “retinoids” encompasses both natural retinoids (retinal, retinoic acid, retinol, retinyl esters) and synthetic analogues, such as adapalene and tretinoin, and provitamin A carotenoids, such as β-carotene.

The chemical structure of retinol is characterized by three structural motifs, a non-aromatic ring structure (β-ionone ring), a polyunsaturated side chain, and an alcohol as the functional end-group. The conjugated double bonds present in retinoid molecules enable retinoids to absorb in the UV-VIS region (325-380 nm). The conjugated double bonds are also the reason why retinoids are thermolabile, photosensitive, and susceptible to oxidation. The structural units of retinal, retinoic acid, and retinyl esters are similar to that of retinol, with the terminal alcohol at the end of the side chain being either an aldehyde in retinal, a carboxylic acid in retinoic acid, or an ester in retinyl esters.

### B. Use of the Compositions

The compositions of the present invention can be used to treat one or more skin conditions. Skin conditions include fine lines or wrinkles, discolored skin (e.g., age spots or dark spots, senile purpura, keratosis, melasma, hyperpigmentation), acne, symptoms associated with acne (e.g., presence of open or closed comedones, papules, pustules, nodulocystic lesions, skin redness, etc.), pruritus, spider veins, lentigo, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, impetigo, erysipelas, erythrasma, eczema, and other inflammatory skin conditions. In certain non-limiting aspects, the skin condition can be caused by exposure to UV light, age, irradiation, chronic sun exposure, environmental pollutants, air pollution, wind, cold, heat, chemicals, disease pathologies, smoking, or lack of nutrition. The skin can be facial skin or non-facial skin (e.g., arms, legs, hands, chest, back, feet, etc.). In particular embodiments, the skin condition can be fine lines or wrinkles, discolored skin, hyperpigmented skin, uneven skin tone, acne, oily skin, dry skin, irritated skin, skin inflammation, loose skin or reduced elasticity of skin, or reddened, rosacea, or erythemic skin.

The compositions of the present invention are used to deliver active ingredients to skin. The active ingredients include one or more retinoids, and can optionally include any additional ingredient that is capable of providing a benefit to the skin.

The compositions can be applied to skin with the condition to be treated, including skin with a fine line or wrinkle, or to hyperpigmented or discolored skin, or to skin having uneven skin tone. The compositions may include one or more additional active ingredients. The one or more additional active ingredients may be selected to treat a variety of skin conditions, including but not limited to acne, dry skin, oily skin, or reddened or erythemic skin, or to any of the aforementioned skin conditions listed in the above paragraphs.

### C. Emulsion Compositions

In certain embodiments, the compositions disclosed herein are provided as emulsions. In particular embodiments, the compositions disclosed herein are provided as oil-in-water emulsions.

The water phase can include ingredients that are known to those of ordinary skill in the art (see, e.g., International Cosmetic Ingredient Dictionary, 11^{th} Ed., 2006) and those that are disclosed throughout the specification. Non-limiting examples of such ingredients include water, polyols, hydrophilic cosmecutical and/or pharmaceutical ingredients, etc., and mixtures thereof. The concentration ranges of these ingredients can vary as explained in other sections of this specification.

The oil phase can also include ingredients that are known to those of ordinary skill in the art (see, e.g., International Cosmetic Ingredient Dictionary, 11^{th} Ed., 2006) and those that are disclosed throughout the specification. Non-limiting examples of such ingredients include oils, fatty acids, fatty alcohols, waxes of natural or synthetic origin, hydrocarbon solvents, film formers, silicones, silicone polymers, fluorinated solvents, etc. The concentration ranges of these ingredients can vary as explained in other sections of this specification. Non-limiting examples of oils that can be used in the context of the present invention include: oils of plant origin (e.g., sweet almond oil, coconut oil, castor oil, jojoba oil, olive oil, rapeseed oil, groundnut oil, sunflower oil, wheat germ oil, maize germ oil, soy based oil, cotton oil, lucerne oil, poppy oil, marrow oil, evening primrose oil, millet oil, barley oil, rye oil, safflower oil, canelle nut tree oil, passionflower oil, hazelnut oil, palm oil, shea butter, apricot stone oil, Alexandria laurel tree oil, sysymbrium oil, avocado oil, calendula oil, etc.); modified plant oils (e.g., products known under INCI designations Apricot Kernel Oil PEG-6 esters, Olive Oil PEG-6 esters, etc.); oils of natural origin (e.g., perhydrosqualene, squalene, etc.); mineral oils (e.g., liquid paraffin, mineral oils originating from petroleum fractions such as isoparaffins having a boiling point between 300 and 400° C., etc.); synthetic oils (e.g., fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate, ester derivatives of lanolic acid, such as isopropyl lanolate, isocetyl lanolate, monoglycerides, triglycerides such as glycerol triheptanoate, alkylbenzoates, isoparaffins, polyalphaolefins, polyolefins, such as polyisobutylene, synthetic isoalkanes such as isohexadecane, isododecane, perfluorinated oils, silicone oils, etc.). Non limiting examples of silicone oils include dimethyl polysiloxanes, methylphenylpolysiloxanes, amine-modified silicones, fatty acid-modified silicones, alcohol-modified silicones, alcohol-and fatty acid-modified silicones, polyether group-modified silicones, epoxy-modified silicones, fluoro group-modified silicones, cyclic silicones, alkyl group-modified silicones, etc. In certain aspects, the silicone oil can be selected from the group consisting of aryl silicones, dimethicone copolyols, cyclic structured siloxanes, dimethicones, low molecular weight alkanes, low molecular weight esters, short chain siloxanes, and silicone acrylates. In certain aspects, the silicone oil is a polyorganosiloxane selected from the group consisting of dimethicone, cyclomethicone, polysilicones, phenyl trimethicone, trimethylsilylamodimethicone, stearoxytrimethylsilane, or mixtures of thereof. In other aspects, the silicone oil can be a volatile silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e., dimethicones having a viscosity of about 50 cst or less (e.g., dimethicones such as Dow Corning 200-0.5 cst Fluid). The Dow Corning Fluids are available from Dow Corning Corporation, Midland, Michigan. Cyclomethicone and dimethicone are described in International Cosmetic Ingredient Dictionary, 11^{th} edition, 2006 as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Michigan.

The aqueous phase and/or the oil phase, prior to emulsification, can include at least one emulsifier. A co-emulsifier can also be used. Emulsifier molecules have a hydrophobic/lipophilic balance (“HLB”) value, i.e., the balance between the hydrophilic and lipophilic portions of the molecule. An emulsifier molecule can be selected based on its HLB value, which affects its ability to control various properties of the emulsion composition. Larger HLB values are associated with emulsifier molecules that are more soluble in water and are better-suited as oil-in-water emulsifiers. Smaller HLB values are associated with emulsifier molecules that are more soluble in oil and are better-suited as water-in-oil emulsifiers. In certain aspects, the HLB value of the emulsifiers in the can be from about 10 to about 19 (including 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0). In other aspects, the HLB value can extend outside this range, as needed. Non-limiting examples of such emulsifiers include taurate, sorbitol, or sorbitan containing emulsifiers (see, e.g., International Cosmetic Ingredient Dictionary, 11th Ed., 2006). Also contemplated are combinations of different taurate containing emulsifiers, combinations of different sorbitol containing emulsifiers, combination of different sorbitan containing emulsifiers, or combinations of taurate, sorbitol, and sorbitan containing emulsifiers. Non-limiting examples of sorbitol and sorbitan containing emulsifiers include Disodium Hydroxydecyl Sorbitol Citrate, Glyceryl/Sorbitol Oleate/Hydroxystearate, Hydroxyethyl Sorbitol, PEG-3/PPG-2 Glyceryl/Sorbitol Hydroxystearate/Isostearate, Polyoxypropylene Sorbitol Ricinoleate, PPG-8 Sorbitol Castor Oil, Rapeseed Oil Sorbitol Esters, Sunflower Seed Oil Sorbitol Esters, Anhydrosorbitol Trioleate, Anhydrosorbitol Tristearate, Dibenzalsorbitol, Lauric Acid, Sorbitol Monoester, PEG-20 Sorbitol Pentaisostearate, PEG-30 Sorbitol Pentaisostearate, PEG-40 Sorbitol Pentaisostearate, PEG-50 Sorbitol Pentaisostearate, Diglyceryl Sorbitan Tetraoctanoate, Fatty Acids, Olive, Monoesters with Sorbitan, Oleic Acid, Diester with Sorbitan, PEG-2 Sorbitan Beeswax, PEG-6 Sorbitan Beeswax, PEG-8 Sorbitan Beeswax, PEG-20 Sorbitan Beeswax, Polyethylene Glycol (100) Sorbitan Beeswax, Polyethylene Glycol 300 Sorbitan Beeswax, and Polyethylene Glycol 400 Sorbitan Beeswax. Non limiting examples of taurate containing emulsifiers include Acrylamide/Sodium Acryloyldimethyltaurate/Acrylic Acid Copolymer, Acrylamide/Sodium Acryloyldimethyltaurate Copolymer, Ammonium Acryloyldimethyltaurate/Beheneth-25 Methacrylate Crosspolymer, Ammonium Acryloyldimethyltaurate/Laureth-7 Methacrylate Copolymer, Ammonium Acryloyldimethyltaurate/Steareth-8 Methacrylate Copolymer, Ammonium Acryloyldimethyltaurate/Steareth-25 Methacrylate Crosspolymer, Ammonium Acryloyldimethyltaurate/Vinyl Formamide Copolymer, Ammonium Acryloyldimethyltaurate/VP Copolymer, Ammonium Polyacryloyldimethyl Taurate, Calcium Lauroyl Taurate, Dimethylacrylamide/Sodium Acryloyldimethyltaurate Crosspolymer, Hydroxyethyl Acrylate/Sodium Acryloyldimethyl Taurate Copolymer, and Potassium Methyl Cocoyl Taurate.

The aqueous phase and/or the oily phase can include a thickening agent. Thickening agents include substances that can increase the viscosity of its solute. Non-limiting examples include those known in the art (e.g., U.S. Pat. Nos. 5,087,445; 4,509,949; 2,798,053; International Cosmetic Ingredient Dictionary, 11^{th} Ed., 2006). Specific examples include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Examples of commercially available carboxylic acid polymers include carbomers, which are homopolymers of acrylic acid crosslinked with allyl ethers of sucrose or pentaerytritol (e.g., Carbopol™ 900 series from B. F. Goodrich). An example of a polysaccharide is guar, which is a polysaccharide with alternating mannose and galactose units. An example of a gum is gum arabic, which can be obtained from the Acacia Senegal tree. Gum arabic forms a thickening gel upon contact with water.

In non-limiting aspects, the composition can be a cosmetic composition. The cosmetic composition can be those described in other sections of this specification or those known to a person of skill in the art. Non-limiting examples of cosmetic compositions include personal care compositions, skin compositions, skin creams, skin gels, skin ointments, skin lotions, skin serums, anti-aging compositions, skin rejuvenation compositions, skin conditioners, moisturizers, skin masks, lipsticks, mascaras, rouges, foundations, blushes, eyeliners, lip liners, lip glosses, lip balms, sunscreens, sunblocks, nail polishes, styling gels, nail conditioners, bath and shower gels, shampoos, conditioners, cream rinses, hair dyes and coloring compositions, soaps, body scrubs, exfoliants, astringents, depilatories and permanent waxing solutions, antidandruff formulations, anti-sweat and antiperspirant compositions, shaving, pre-shaving and after shaving products, perfumes, colognes, leave-on conditioners, deodorants, cold creams, deodorants, toners, cleansers, rinses, or the like; whether in the form of creams, lotions, gels, ointments, macro-emulsions, micro-emulsions, nano-emulsions, serums, balms, colloids, solutions, liquids, suspensions, dispersions, brush-on formulations, skin delivery enhancing systems, masks, pastes, or the like.

The composition can have a dermatologically acceptable vehicle or carrier for a plant, plant part, or extract thereof. The composition can further include a moisturizing agent or a humectant, a surfactant, a silicone containing compounds, a UV agent, an oil, and/or other ingredients identified in this specification or those known in the art. The compositions of the present invention can also include any one of, any combination of, or all of the following additional ingredients: water, a chelating agent, a moisturizing agent, a preservative, a thickening agent, a silicone containing compound, an essential oil, a structuring agent, a vitamin, a pharmaceutical ingredient, or an antioxidant, or any combination of such ingredients or mixtures of such ingredients. In certain aspects, the composition can include at least two, three, four, five, six, seven, eight, nine, ten, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference. The compositions can be storage stable or color stable, or both.

### D. Emulsion Structure and Preparation

In some embodiments, the methods disclosed herein are employed for the production of emulsions that have been prepared by the emulsification of an aqueous or water phase and an oil phase. In certain embodiments, the emulsions are oil-in-water emulsions.

The emulsions can be prepared by using techniques that are known in the art (see, e.g., Mitxhell and Schlossman, The Chemistry and Manufacture of Cosmetics: Volume II-Formulating, 2000; Volume II Formulating, Chapter 7, pages 135-150). For instance, an emulsion can be made by combining an aqueous phase, an oily phase, and, optionally an emulsifier and/or a thickening agent, and homogenizing the mixture by using standard homogenization procedures, which results in the formulation of the emulsion. The homogenization procedure can be performed at room temperature (e.g., approximately 25° C.) without heating either the aqueous phase or the oil phase, prior, during, or after the homogenization. In some embodiments, at least one of the oil phase and the water phase is heated prior to homogenization. In some embodiments, the homogenizing mixture is heated during homogenization. In some embodiments, the homogenized mixture is heated after homogenization. Another aspect of the homogenization procedure is that the aqueous phase can be added to the oil phase prior to or during mixing or the oil phase can be added to the aqueous phase prior to or during mixing.

The viscosity can be modified to a desired range by adding and adjusting the concentration of a thickening agent in the water and/or oil phase. There are many available methods that can be used to determine the viscosity of any given composition, including phases of an emulsion. For instance, viscosity of an emulsion, any phase within an emulsion, or any composition that includes the emulsion can be determined by using a T spindle at 2.5 rpm at room temperature (e.g., approximately 25° C.). A Brookfield Viscometer/Rheometer can be used. In certain aspects, the viscosity of an oily phase or a water phase is between 50,000 to 150,000 cps at 25° C. as measured on a Brookfield Viscometer using a TC spindle at 2.5 rpm. However, as explained elsewhere in this specification, the viscosity range can vary inside or outside this range, as desired. It is also contemplated that the viscosity of the composition can be selected to achieve a desired result, e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps, etc., as measured on a Brookfield Viscometer using a TC spindle at 2.5 rpm at 25° C.).

### E. Chemical Definitions

The term “ester” refers to a chemical moiety with formula —COOR, where R is selected from among alkyl, cycloalkyl, aryl, heteroaryl (bonded through a ring carbon) and heterocycloalkyl (bonded through a ring carbon). In some embodiments, any hydroxy, or carboxyl side chain on the compounds described herein is esterified.

The term “alkyl” as used herein, means a straight, branched chain, or cyclic (in this case, it would also be known as “cycloalkyl”) hydrocarbon containing from 1-10 carbon atoms. Illustrative examples of alkyl include, but are not limited to, methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl.

The term “aryl” as used herein, refers to an aromatic ring wherein each of the atoms forming the ring is a carbon atom. In some embodiments, aryl rings are formed by five, six, seven, eight, nine, or more than nine carbon atoms. Examples of aryl groups include, but are not limited to phenyl, naphthalenyl, phenanthrenyl, anthracenyl, fluorenyl, and indenyl.

The term “heteroaryl” means heteroaryl groups that are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkylene, mercapto, nitro, —NRARB, and —RARB)carbonyl.

### F. Additional Ingredients

Compositions of the present invention can include additional ingredients. Non-limiting examples of additional ingredients include cosmetic ingredients and pharmaceutical active ingredients.

### 1. Cosmetic Ingredients

The CTFA International Cosmetic Ingredient Dictionary and Handbook (2006) describes a wide variety of non-limiting cosmetic ingredients that can be used in the context of the present invention. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, co-emulsifiers, stabilizers, lubricants, solvents, moisturizers (including, e.g., emollients, humectants, film formers, occlusive agents, and agents that affect the natural moisturization mechanisms of the skin), water-repellants, UV absorbers (physical and chemical absorbers such as titanium dioxide, zinc oxide, avobenzone, octocrylene, benzophenone, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., glycerin, propylene glycol, butylene glycol, pentylene glycol, sorbitol, urea, and manitol), exfoliants (e.g., alpha-hydroxyacids, and beta-hydroxyacids such as lactic acid, glycolic acid, and salicylic acid; and salts thereof) waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate). Non-limiting examples of some of these ingredients are provided in the following subsections.

a. Co-Emulsifiers

Compositions of the present invention can also include a co-emulsifier. A co-emulsifier can include surfactants that can be used in combination with the emulsifiers disclosed in the present invention to form stable emulsions. Non-limiting examples of surfactants that can be used include nonionic, cationic, anionic, and zwitterionic surfactants (See McCutcheon's (1986); U.S. Pat. Nos. 5,011,681; 4,421,769; 3,755,560). For instance, examples of co-emulsifiers include esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof (See International Cosmetic Ingredient Dictionary, 11^{th} Ed., 2006). It is also contemplated that certain co-emulsifiers, including one or more of the agents disclosed herein, can be excluded from compositions of the present invention.

b. Stabilizers

Emulsions of the present invention can also include a stabilization agent. Stabilization agents can be an ingredient that aids in stabilizing emulsions or compositions of the present invention. Non-limiting examples of stabilization agents include those known to a person of ordinary skill in the art (see, e.g., International Cosmetic Ingredient Dictionary, 11^{th} Ed., 2006) and those disclose throughout the specification. Examples include hydrogenated castor oil, plant waxes (e.g., beeswax and carnauba wax, etc.), stearic acid, magnesium stearate, aluminum stearate, hydrophobic silicas, polyethylene glycol-alkyl glycol copolymers, mineral waxes, etc.

However, it is noted that stabilization agents are not required in the context of the present invention to obtain a stable composition. Indeed, as described above, in some embodiments the compositions disclosed herein do not include stabilization agents.

c. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal, chlorophensin, Chlorohexedine Digluconate, DMDM Hydantion, Iodopropylbutylcarbamate, Hexetidine, Dichorobenzyl Alcohol, Methyldibromoglutaronitrile or combinations thereof. It is also contemplated that certain preservatives, including one or more of the preservatives disclosed herein, can be excluded from compositions of the present invention.

d. Moisturizers

Non-limiting examples of moisturizing agents that can be used with the compositions of the present invention can be found in the International Cosmetic Ingredient Dictionary, 11^{th} Ed., 2006. Examples include include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol. It is also contemplated that certain moisturizing agents, including one or more of the agents disclosed herein, can be excluded from compositions of the present invention.

e. Emollients

Non-limiting examples of emollients include, but are not limited to, vegetable oils, mineral oils, silicone oils, synthetic and natural waxes, medium chain triglycerides, petrolatum, lanolin, aluminum magnesium hydroxide stearate (which can also function as a water repellent), and fatty acid esters. Non-limiting examples of vegetable oils include safflower oil, corn oil, sunflower seed oil, and olive oil. It is also contemplated that certain emollients, including one or more of the emollients disclosed herein, can be excluded from compositions of the present invention.

f. Antioxidants

Non-limiting examples of antioxidants include, but are not limited to, acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite. It is also contemplated that certain antioxidants, including one or more of the antioxidants disclosed herein, can be excluded from compositions of the present invention.

g. Colorants

In certain non-limiting aspects, a colorant can be dispersed throughout the compositions and/or a phase (e.g., water or oil phase) of the composition. Non-limiting examples of colorants that can be used in the context of the present invention include those known to a person of ordinary skill in the art (see, e.g., CTFA International Cosmetic Ingredient Dictionary and Handbook (2006)). For instance natural and synthetic pigments and lakes can be used. Examples of groups of pigments include carbon, cadmium, iron oxide, Prussian blue, chromium, cobalt, copper, titanium, ultramarine, zinc, clay earth, and organic pigments. Specific non-limiting examples of colorants include Aluminum Powder, Blue 1 Lake, Bronze Powder, Chromium Oxide Greens, Copper Powder, Ext. Yellow 7 Lake, Green 3 Lake, Orange 4 Lake, Orange 5 Lake, Orange 10 Lake, Pigment Blue 15, Pigment Blue 15:2, Pigment green 7, Pigment Orange 5, Pigment Red 4, Pigment Red 5, Pigment Red 48, Pigment Red 53, Pigment Red 53:1, Pigment Red 57, Pigment Red 57:1, Pigment Red 63:1, Pigment Red 64:1, Pigment Red 68, Pigment Red 83, Pigment Red 88, Pigment Red 90:1 Aluminum Lake, Pigment Red 112, Pigment Red 172 Aluminum Lake, Pigment Red 173 Aluminum Lake, Pigment Red 190, Pigment Violet 19, Pigment Yellow 1, Pigment Yellow 3, Pigment Yellow 12, Pigment Yellow 13, Pigment Yellow 73, Red 4 Lake, Red 6 Lake, Red 7 Lake, Red 21 Lake, Red 22 Lake, Red 27 Lake, Red 28 Lake, Red 30 Lake, Red 31 Lake, Red 33 Lake, Red 34 Lake, Red 36 Lake, Red 40 Lake, Sunset Yellow Aluminum Lake, Yellow 5 Lake, Yellow 6 Lake, Yellow 7 Lake, Yellow 10 Lake, and Zinc Oxide. It is also contemplated that certain colorants, including one or more of the colorants disclosed herein, can be excluded from compositions of the present invention.

### 2. Pharmaceutical Active Agents

Pharmaceutical active agents are also contemplated as being useful with the compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### G. Amount of Ingredients

A person of ordinary skill would recognize that the compositions of the present invention can include any number of combinations of ingredients identified above and throughout this specification discussed throughout this specification. The concentrations of the ingredients can vary. In non-limiting embodiments, for example, the emulsion compositions may include in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or more, or any range or integer derivable therein, of at least one of the ingredients mentioned throughout the specification and claims. In non-limiting aspects, the percentage of the ingredients can be calculated by weight or volume of the total weight of a cosmetic or pharmaceutical product that includes the compositions disclosed herein. A person of ordinary skill in the art would understand that the concentrations can vary depending on the desired effect of the composition into which a compound is incorporated.

### H. Equivalents

Known and unknown equivalents to the ingredients discussed throughout this specification can be used with the emulsion compositions and methods of the present invention. The equivalents can be used as substitutes for the ingredients. The equivalents can also be used to add to the methods and emulsions of the present invention. A person of ordinary skill in the art would be able to recognize and identify acceptable known and unknown equivalents to the ingredients without undue experimentation.

### I. Products

The compositions of the present invention can be incorporated into products. Non-limiting examples of products include cosmetic products, food-based products, pharmaceutical products, etc. By way of example only, non-limiting cosmetic products include sunscreen products, sunless skin tanning products, hair products, fingernail products, moisturizing creams, skin benefit creams and lotions, softeners, day lotions, gels, ointments, foundations, night creams, lipsticks, mascaras, eyeshadows, eyeliners, cheek colors, cleansers, toners, masks, or other known cosmetic products or applications. Additionally, the cosmetic products can be formulated as leave-on or rinse-off products.

### J. Kits

Kits are also contemplated as being used in certain aspects of the present invention. For instance, an emulsion of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of an emulsion. In other embodiments, the container can be squeezed (e.g., metal, laminate, or plastic tube) to dispense a desired amount of an emulsion as disclosed herein. The composition can be dispensed as a liquid, a fluid, or a semi-solid. The containers can have spray, pump, or squeeze mechanisms. A kit can also include instructions for using the kit and/or emulsion. Instructions can include an explanation of how to apply, use, and maintain the compositions

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### (Exemplary Formulation)

A formulation having the components listed in Table 1 were prepared as a topical skin composition. Components A and B were placed in a beaker and degassed with argon gas until less than 1.0 ppm of oxygen was detected. The beaker was heated to 80° C. while adding a component C and mixing to provide an intermediate comprising components A, B, and C (intermediate ABC). Component D was prepared in a separate beaker and heated to 80° C. while mixing. Component D was added to intermediate ABC while mixing at 80° C. to form an emulsion. The emulsion was homogenized at 10k RPM for 1 min. The emulsion was allowed to cool to room temperature while stirring. Component F was added to the beaker and mixed. Component G was used to adjust the pH to between 6 and 7. Component H was added to the vessel and stirred. The solution was homogenized at 10k RPM for 1 min. Component I was added and stirred until homogenous. The mixing was carried out under inert gas. The resulting lotion was placed in foil lined airless pumps and blanketed with argon gas prior to sealing. Samples were stored at 25° C. and 40° C. for accelerated aging studies. Exemplary amount (concentration) ranges of each component are included in Table 1.

TABLE 1
Exemplary formulation

| | | % Concentration |
|---|---|---|
| Component | Ingredient | (by weight) |
| A | Water | 5.0-95 |
| A | Citrate-phosphate buffer (10% solution) | 0.01-5.0 |
| | pH = 5 | |
| B | Disodium EDTA | 0.01-5.0 |
| B | Optiphen | 1.0 |
| C | Glycerin | 1.0-5.0 |
| C | Xanthan gum | 0.1-0.2 |
| D | Dimethicone | 1.0-10.0 |
| D | Neopentyl glycol diheptanoate | 1.0-10.0 |
| D | Isononyl isononanoate | 1.0-10.0 |
| D | Tocopheryl acetate | 0.01-5.0 |
| D | Butylated hydroxytoluene | 0.01-5.0 |
| D | Glyceryl stearate/PEG-100 stearate | 1.0-5.0 |
| D | Ceteareth-33 | 0.01-1.0 |
| F | Ascorbic acid | 0.01-5.0 |
| G | KOH 45% solution | Adjust pH |
| | | to 6-7 |
| H | Retinol & Polysorbate 20 | 0.01-5.0 |
| I | Aristoflex AVC | 0.01-3.0 |

### Example 2

### (Exemplary Formulation)

A formulation having the components listed in Table 1 were prepared as a topical skin composition. Component A was placed in a beaker and mixed until uniformity was reached. Component B was added to component A and the resulting mixture was heated to 75-80° C. while adding a component C and mixing to provide an intermediate comprising components A, B, and C (intermediate ABC). Component D was prepared in a separate beaker, heated to 75-80° C., then added to intermediate ABC while mixing at 75-80° C. to form an emulsion (ABCD). Component E was added to emulsion ABCD then the combined mixture (ABCDE) homogenized at 6k RPM for 5 minutes. The emulsion was allowed to cool to 50° C. Component F was added to the cooled emulsion at 50° C., and the resulting mixture (ABCDEF) was stirred for 2 minutes, then homogenized form 3 minutes at 6K RPM. Mixture ABCDEF was then cooled to 30° C. Component G was added to mixture ABCDEF at° C., then component H was added to the mixture. Component I was warmed to 45° C., then added to the mixture, followed by mixing for 5 minutes. Component J was added to the mixture, then mixed for 10 minutes, followed by homogenization at 6k RPM. The mixture was then cooled to 30-33° C. then placed in a flask. Air was evacuated from the flask then replaced with argon gas and scaled. Exemplary amount (concentration) ranges of each component are included in Table 2.

TABLE 2
Exemplary Formulation

| | | % Concentration |
|---|---|---|
| Component | Ingredient | (by weight) |
| A | Water | 5.0-95.0 |
| A | Disodium phosphate | 0.01-5.0 |
| A | Citric acid | 0.01-5.0 |
| B | Hydroxyethyl urea | 1.0-5.0 |
| B | Disodium EDTA | 0.01-5.0 |
| B | Hydroxyacetophenone | 0.1-0.5 |
| B | Caprylyl glycol and phenoxyethanol | 1.0 |
| | (Optiphen) | |
| C | Glycerin | 1.0-5.0 |
| C | Xanthan Gum | 0.1-0.5 |
| D | Neopentyl glycol diheptanoate | 1.0-10.0 |
| D | Isononyl isononanoate | 1.0-10.0 |
| D | Tocopheryl acetate | 0.1-0.5 |
| D | Glyceryl stearate/PEG-100 stearate | 1.0-5.0 |
| D | Ceteareth-33 | 0.1-2.0 |
| E | Non-porous silica | 1.0-3.0 |
| E | Silica-highly porous, ultrafine | 1.0-5.0 |
| F | Methyl trimethicone | 1.0-10.0 |
| F | Silica silylate (Dowsil VM-2270 | 0.1-0.5 |
| | Aerogel) | |
| G | Water | 1.00 |
| G | Ascorbic acid | 0.1-5.0 |
| H | Water | 1.00 |
| H | KOH 45% solution | Adjust pH |
| | | to 6-7 |
| I | Retinol & Polysorbate 20 | 0.1-5.0 |
| J | Aristoflex AVC | 0.1-3.0 |

### Example 3

### (Exemplary Formulation)

A formulation having the components listed in Table 1 were prepared as a topical skin composition. Exemplary amount (concentration) ranges of each component are included in Table 3.

TABLE 3
Exemplary Formulation

| | % Concentration |
|---|---|
| Ingredient | (by weight) |
| Water | 5.0-95.0 |
| Isononyl isononanoate | 1.0-10.0 |
| Methyl trimethicone | 1.0-10.0 |
| Neopentyl glycol diheptanoate | 1.0-10.0 |
| Glycerin | 1.0-10.0 |
| Silica | 1.0-10.0 |
| Ammonium acryloyldimethyltaurate/VP copolymer | 0.1-5.0 |
| Glyceryl Stearate | 0.1-5.0 |
| PEG-100 Stearate | 0.1-5.0 |
| Hydroxyethyl urea | 0.1-5.0 |
| Ceteareth-33 | 0.1-5.0 |
| Tocopheryl acetate | 0.1-5.0 |
| Phenoxyethanol | 0.1-5.0 |
| Polysorbate-20 | 0.1-5.0 |
| Caprylyl glycol | 0.1-5.0 |
| Disodium phosphate | 0.1-0.5 |
| Retinol | 0.1-0.5 |
| Hydroxyacetophenone | 0.1-0.5 |
| Citric acid | 0.1-0.5 |
| Disodium EDTA | 0.1-0.5 |
| Potassium hydroxide | 0.1-0.5 |
| Xanthan gum | 0.1-0.5 |
| Ascorbic acid | 0.01-0.5 |
| Silica silylate | 0.01-0.2 |
| Ammonium lactate | 0.01-0.1 |

### Example 4

### (Accelerated Aging Studies)

A variety of tests can be used to determine if the compositions exhibit retinoid-stabilizing effects. One example of such a test is a long term stability program at elevated temperatures for twelve (12) weeks with assessments for instability (e.g., check for phase separation of the emulsion) at four (4) week intervals. Another example of such a test is a pH-maintenance test at elevated temperatures for extended periods of time in order to assess the pH-maintaining abilities of different buffers.

Tables 4-6 below include stability data from accelerated aging studies of retinoid-containing compositions using different buffers at different concentrations and pH values. The studies examined retinoid stability at different temperatures and different time periods. The studies employed high performance liquid chromatography (HPLC) to determine the fractions of original retinoid remaining after the accelerated aging studies. The buffer was added at 0.33, 0.66, and 1.0 wt. %, relative to the weight of the composition. The compositions were maintained at 25° C. or 40° C. for 8 or 12 weeks. Each experiment was repeated four times (trials), and the average (and standard deviation) for each set of four trials is included.

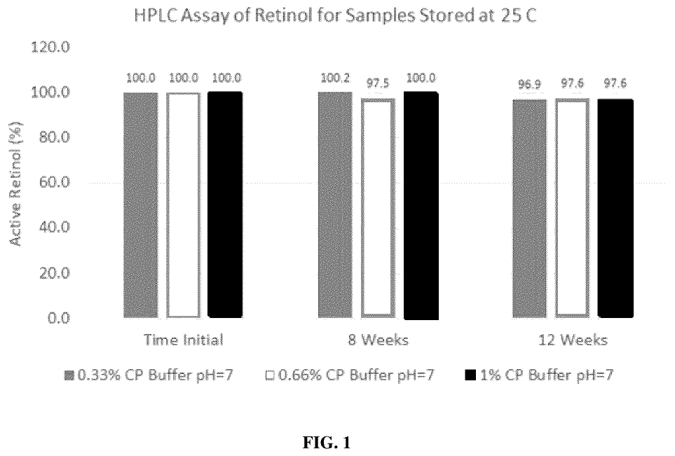

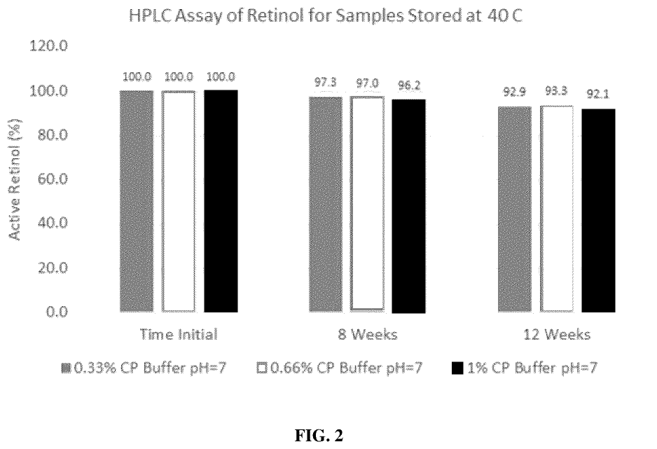

In Table 4, the retinoid-stabilizing effect of a citrate-phosphate buffer at a pH value of 7 was examined. The results are depicted graphically in FIG. 1 and FIG. 2.

TABLE 4
HPLC assay of retinol cream in citrate-phosphate buffer pH = 7

| | | | Retinol % | | | | |
|---|---|---|---|---|---|---|---|
| | Buffer | | | 8 Weeks | | 12 Weeks | |
| Experiment # | (wt %) | Trial | Initial | 25° C. | 40° C. | 25° C. | 40° C. |
| 1 | 0.33 | 1 | 0.6785 | 0.6826 | 0.6534 | 0.6623 | 0.6521 |
| | | 2 | 0.6781 | 0.6814 | 0.6533 | 0.6614 | 0.6243 |
| | | 3 | 0.66722 | 0.6703 | 0.6557 | 0.6520 | 0.6293 |
| | | 4 | 0.6718 | 0.6707 | 0.6553 | 0.6522 | 0.6297 |
| | | AVG | 0.6752 | 0.6763 | 0.6544 | 0.6570 | 0.6271 |
| | | STD | 0.0036 | 0.0067 | 0.0013 | 0.0056 | 0.0028 |
| | | DEV | | | | | |
| 2 | 0.66 | 1 | 0.6872 | 0.6764 | 0.6645 | 0.6659 | 0.6414 |
| | | 2 | 0.6865 | 0.6763 | 0.6644 | 0.6655 | 0.6407 |
| | | 3 | 0.6774 | 0.6544 | 0.6676 | 0.6583 | 0.6323 |
| | | 4 | 0.6773 | 0.6543 | 0.6674 | 0.6579 | 0.316 |
| | | AVG | 0.6821 | 0.6654 | 0.6660 | 0.6619 | 0.6365 |
| | | STD | 0.0055 | 0.0127 | 0.0018 | 0.0044 | 0.0053 |
| | | DEV | | | | | |
| 3 | 1.00 | 1 | 0.6600 | 0.6531 | 0.6469 | 0.6364 | 0.6226 |
| | | 2 | 0.6602 | 0.6525 | 0.6464 | 0.6359 | 0.6222 |
| | | 3 | 0.6611 | 0.6693 | 0.6436 | 0.6344 | 0.5944 |
| | | 4 | 0.6617 | 0.6683 | 0.6425 | 0.6352 | 0.5947 |
| | | AVG | 0.6608 | 0.6608 | 0.6449 | 0.6355 | 0.6085 |
| | | STD | 0.0092 | 0.0092 | 0.0021 | 0.0009 | 0.0161 |
| | | DEV | | | | | |

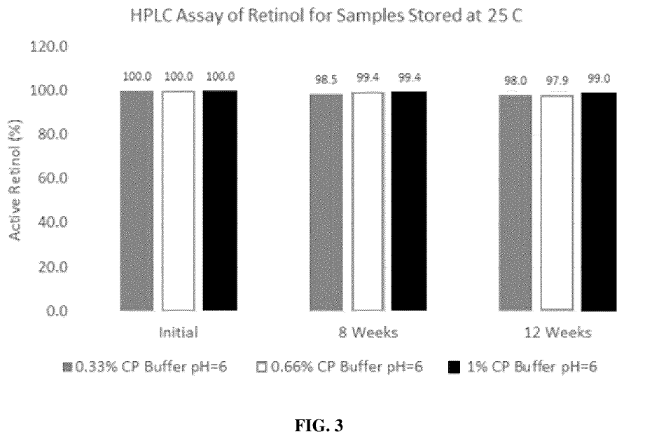

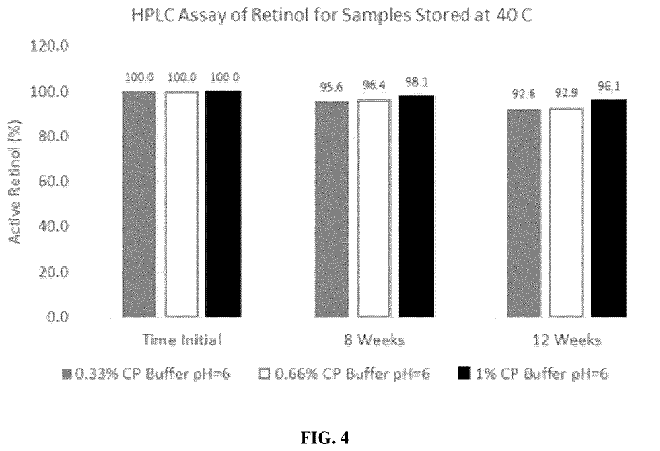

In Table 5, the retinoid-stabilizing effect of a citrate-phosphate buffer at a pH value of 6 was examined. The results are depicted graphically in FIG. 3 and FIGS. 4.

TABLE 5
HPLC assay of retinol cream comprised of citrate-phosphate buffer pH = 6

| | | | Retinol % | | | | |
|---|---|---|---|---|---|---|---|
| | Buffer | | | 8 Weeks | | 12 Weeks | |
| Experiment # | (wt %) | Trial | Initial | 25° C. | 40° C. | 25° C. | 40° C. |
| 1 | 0.33 | 1 | 0.6436 | 0.6290 | 0.6100 | 0.6319 | 0.5988 |
| | | 2 | 0.6424 | 0.6280 | 0.6100 | 0.6310 | 0.5982 |
| | | 3 | 0.6443 | 0.6390 | 0.6210 | 0.6292 | 0.5933 |
| | | 4 | 0.6439 | 0.6390 | 0.6200 | 0.6296 | 0.5932 |
| | | AVG | 0.6436 | 0.6338 | 0.6153 | 0.6304 | 0.5959 |
| | | STD | 0.0008 | 0.0061 | 0.0061 | 0.0013 | 0.0030 |
| | | DEV | | | | | |
| 2 | 0.66 | 1 | 0.6181 | 0.6170 | 0.6020 | 0.6092 | 0.5675 |
| | | 2 | 0.6172 | 0.6160 | 0.6010 | 0.6090 | 0.5675 |
| | | 3 | 0.6195 | 0.6180 | 0.5950 | 0.6050 | 0.5849 |
| | | 4 | 0.6270 | 0.6170 | 0.5940 | 0.6058 | 0.5846 |
| | | AVG | 0.6205 | 0.6170 | 0.5980 | 0.6073 | 0.5761 |
| | | STD | 0.0045 | 0.0008 | 0.0041 | 0.0022 | 0.0100 |
| | | DEV | | | | | |
| 3 | 1.00 | 1 | 0.6150 | 0.6090 | 0.6030 | 0.6093 | 0.5880 |
| | | 2 | 0.6151 | 0.6090 | 0.6030 | 0.6089 | 0.5871 |
| | | 3 | 0.6140 | 0.6120 | 0.6020 | 0.6074 | 0.5940 |
| | | 4 | 0.6136 | 0.6120 | 0.6020 | 0.6068 | 0.5933 |
| | | AVG | 0.6144 | 0.6105 | 0.6025 | 0.6081 | 0.5906 |
| | | STD | 0.0007 | 0.0017 | 0.0006 | 0.0012 | 0.0036 |
| | | DEV | | | | | |

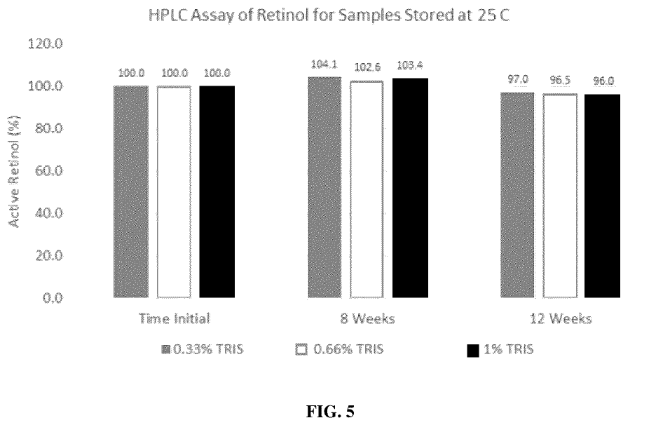

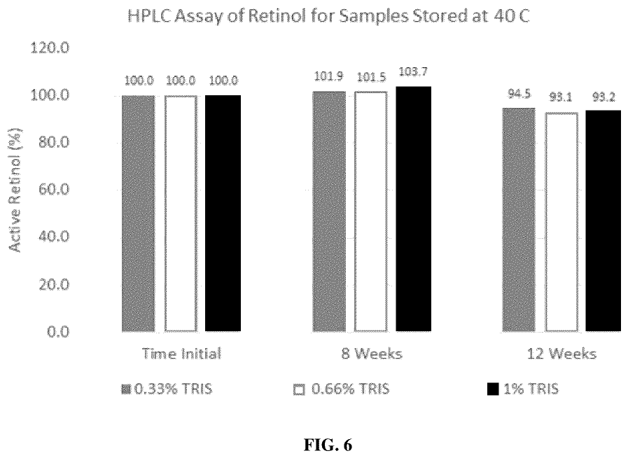

In Table 6, the retinoid-stabilizing effect of a TRIS buffer at a pH value of 7 was examined. The results are depicted graphically in FIG. 5 and FIGS. 6.

TABLE 6
HPLC assay of retinol cream comprised of TRIS buffer pH = 7

| | | | Retinol % | | | | |
|---|---|---|---|---|---|---|---|
| | Buffer | | | 8 Weeks | | 12 Weeks | |
| Experiment # | (wt %) | Trial | Initial | 25° C. | 40° C. | 25° C. | 40° C. |
| 1 | 0.33 | 1 | 0.6220 | 0.6443 | 0.6247 | 0.5960 | 0.5786 |
| | | 2 | 0.6210 | 0.6431 | 0.6228 | 0.5951 | 0.5786 |
| | | 3 | 0.6180 | 0.6377 | 0.6302 | 0.5982 | 0.5843 |
| | | 4 | 0.6180 | 0.6377 | 0.6303 | 0.5971 | 0.5845 |
| | | AVG | 0.6198 | 0.6407 | 0.6270 | 0.5966 | 0.5816 |
| | | STD | 0.0021 | 0.0035 | 0.0038 | 0.0013 | 0.0033 |
| | | DEV | | | | | |
| 2 | 0.66 | 1 | 0.6250 | 0.6439 | 0.6308 | 0.6001 | 0.5849 |
| | | 2 | 0.6250 | 0.6436 | 0.6299 | 0.5991 | 0.5840 |
| | | 3 | 0.6300 | 0.6371 | 0.6363 | 0.6051 | 0.5775 |
| | | 4 | 0.6300 | 0.6363 | 0.6364 | 0.6048 | 0.5771 |
| | | AVG | 0.6275 | 0.6402 | 0.6334 | 0.6023 | 0.5809 |
| | | STD | 0.0029 | 0.0041 | 0.0035 | 0.0031 | 0.0041 |
| | | DEV | | | | | |
| 3 | 1.00 | 1 | 0.6110 | 0.6304 | 0.6263 | 0.5875 | 0.5726 |
| | | 2 | 0.6100 | 0.6297 | 0.6267 | 0.5869 | 0.5730 |
| | | 3 | 0.6140 | 0.6336 | 0.6403 | 0.5857 | 0.5662 |
| | | 4 | 0.6130 | 0.6329 | 0.6393 | 0.5858 | 0.5660 |
| | | AVG | 0.6120 | 0.6317 | 0.6332 | 0.5865 | 0.5695 |
| | | STD | 0.0018 | 0.0019 | 0.0077 | 0.0009 | 0.0039 |
| | | DEV | | | | | |

Maintenance of pH is correlated with retinoid stability. In view of this, Tables 7-12 below include pH data after accelerated aging studies of retinoid-containing compositions. The studies examine pH values after different storage durations (4, 8, and 12 weeks) at increased temperatures (25° C. or 40° C.). The studies also examined the effects of different buffers added at different concentrations. Each experiment was repeated four times (trials), and the average (and standard deviation) for each set of four trials is included.

In Table 7, the pH-maintaining effect of a citrate-phosphate buffer at a pH value of 6 stored at 40° C. was examined.

TABLE 7
pH of retinol cream comprised of citrate-phosphate buffer pH = 6

| | Buffer | | pH | | | |
|---|---|---|---|---|---|---|
| Experiment # | (wt %) | Trial | Initial | 4 w 40° C. | 8 w 40° C. | 12 w 40° C. |
| 1 | 0.33 | 1 | 6.06 | 6.02 | 6.29 | 6.31 |
| | | 2 | 6.04 | 6.10 | 6.27 | 6.39 |
| | | 3 | 6.03 | 6.09 | 6.28 | 6.48 |
| | | 4 | 6.05 | 6.02 | 6.29 | 6.51 |
| | | AVG | 6.05 | 6.06 | 6.28 | 6.52 |
| | | STD | 0.01 | 0.04 | 0.01 | 0.09 |
| | | DEV | | | | |
| 2 | 0.66 | 1 | 6.08 | 6.18 | 6.28 | 6.45 |
| | | 2 | 6.09 | 6.16 | 6.29 | 6.43 |
| | | 3 | 6.09 | 6.17 | 6.24 | 6.42 |
| | | 4 | 6.12 | 6.17 | 6.20 | 6.42 |
| | | AVG | 6.10 | 6.16 | 6.25 | 6.43 |
| | | STD | 0.02 | 0.02 | 0.04 | 0.01 |
| | | DEV | | | | |
| 3 | 1.00 | 1 | 6.15 | 6.23 | 6.26 | 6.42 |
| | | 2 | 6.11 | 6.22 | 6.37 | 6.43 |
| | | 3 | 6.13 | 6.18 | 6.37 | 6.43 |
| | | 4 | 6.12 | 6.20 | 6.38 | 6.39 |
| | | AVG | 6.13 | 6.21 | 6.35 | 6.42 |
| | | STD | 0.02 | 0.02 | 0.06 | 0.02 |
| | | DEV | | | | |

In Table 8, the pH-maintaining effect of a citrate-phosphate buffer at a pH value of 6 stored at 25° C. was examined.

TABLE 8
pH of retinol cream comprised of citrate-phosphate buffer pH = 6

| | Buffer | | pH | | | |
|---|---|---|---|---|---|---|
| Experiment # | (wt %) | Trial | Initial | 4 w 25° C. | 8 w 25° C. | 12 w 25° C. |
| 1 | 0.33 | 1 | 6.06 | 6.12 | 6.20 | 6.17 |
| | | 2 | 6.04 | 6.09 | 6.21 | 6.16 |
| | | 3 | 6.03 | 6.11 | 6.22 | 6.15 |
| | | 4 | 6.05 | 6.08 | 6.21 | 6.17 |
| | | AVG | 6.05 | 6.10 | 3.21 | 6.16 |
| | | STD | 0.01 | 0.02 | 0.01 | 0.01 |
| | | DEV | | | | |
| 2 | 0.66 | 1 | 6.08 | 6.14 | 6.23 | 6.10 |
| | | 2 | 6.09 | 6.15 | 6.25 | 6.24 |
| | | 3 | 6.09 | 6.13 | 6.26 | 6.22 |
| | | 4 | 6.12 | 6.16 | 6.18 | 6.24 |
| | | AVG | 6.10 | 6.15 | 6.23 | 6.20 |
| | | STD | 0.02 | 0.01 | 0.04 | 0.07 |
| | | DEV | | | | |
| 3 | 1.00 | 1 | 6.15 | 6.12 | 6.10 | 6.24 |
| | | 2 | 6.11 | 6.20 | 6.20 | 6.31 |
| | | 3 | 6.13 | 6.24 | 6.22 | 6.32 |
| | | 4 | 6.12 | 6.26 | 6.26 | 6.33 |
| | | AVG | 6.13 | 6.21 | 6.20 | 6.30 |
| | | STD | 0.02 | 0.06 | 0.07 | 0.04 |
| | | DEV | | | | |

In Table 9, the pH-maintaining effect of a citrate-phosphate buffer at a pH value of 7 stored at 40° C. was examined.

TABLE 9
pH of retinol cream comprised of citrate-phosphate buffer
pH = 7 stored at 40° C. for 12 weeks.

| | Buffer | | pH | | | |
|---|---|---|---|---|---|---|
| Experiment # | (wt %) | Trial | Initial | 4 w 40° C. | 8 w 40° C. | 12 w 40° C. |
| 1 | 0.33 | 1 | 7.09 | 6.98 | 7.15 | 7.01 |
| | | 2 | 7.10 | 6.97 | 7.13 | 7.04 |
| | | 3 | 6.99 | 7.03 | 7.12 | 7.09 |
| | | 4 | 6.98 | 6.96 | 7.16 | 7.09 |
| | | AVG | 7.04 | 6.99 | 7.14 | 7.06 |
| | | STD | 0.06 | 0.03 | 0.02 | 0.04 |
| | | DEV | | | | |
| 2 | 0.66 | 1 | 6.98 | 7.04 | 7.16 | 7.10 |
| | | 2 | 6.99 | 7.06 | 7.15 | 7.10 |
| | | 3 | 7.01 | 7.06 | 7.19 | NA |
| | | 4 | 6.96 | 6.99 | 7.18 | NA |
| | | AVG | 6.99 | 7.04 | 7.17 | 7.10 |
| | | STD | 0.02 | 0.03 | 0.02 | 0.00 |
| | | DEV | | | | |
| 3 | 1.00 | 1 | 6.89 | 7.04 | 7.16 | 7.14 |
| | | 2 | 6.93 | 7.05 | 7.19 | 7.14 |
| | | 3 | 6.98 | 7.08 | 7.20 | 7.16 |
| | | 4 | 6.97 | 7.03 | 7.20 | 7.14 |
| | | AVG | 6.94 | 7.05 | 7.19 | 7.15 |
| | | STD | 0.04 | 0.02 | 0.02 | 0.01 |
| | | DEV | | | | |

In Table 10, the pH-maintaining effect of a citrate-phosphate buffer at a pH value of 7 stored at 25° C. was examined.

TABLE 10
pH of retinol cream comprised of citrate-phosphate buffer pH = 7

| | Buffer | | pH | | | |
|---|---|---|---|---|---|---|
| Experiment # | (wt %) | Trial | Initial | 4 w 25° C. | 8 w 25° C. | 12 w 25° C. |
| 1 | 0.33 | 1 | 7.09 | 6.93 | 7.18 | 6.95 |
| | | 2 | 7.10 | 7.04 | 7.16 | 6.98 |
| | | 3 | 6.99 | 7.04 | 7.12 | 7.00 |
| | | 4 | 6.98 | 7.06 | 6.84 | 7.04 |
| | | AVG | 7.04 | 7.02 | 7.08 | 6.99 |
| | | STD | 0.06 | 0.06 | 0.16 | 0.04 |
| | | DEV | | | | |
| 2 | 0.66 | 1 | 6.98 | 7.08 | 7.07 | 7.04 |
| | | 2 | 6.99 | 7.03 | 7.13 | 7.05 |
| | | 3 | 7.01 | 6.98 | 7.14 | 7.04 |
| | | 4 | 6.96 | 7.06 | 7.14 | 7.04 |
| | | AVG | 6.99 | 7.04 | 7.12 | 7.04 |
| | | STD | 0.02 | 0.04 | 0.03 | 0.00 |
| | | DEV | | | | |
| 3 | 1.00 | 1 | 6.89 | 7.09 | 7.20 | 7.10 |
| | | 2 | 6.93 | 7.08 | 7.17 | 7.10 |
| | | 3 | 6.98 | 7.09 | 7.18 | 7.13 |
| | | 4 | 6.97 | 7.10 | 7.18 | 7.13 |
| | | AVG | 6.94 | 7.09 | 7.18 | 7.12 |
| | | STD | 0.04 | 0.01 | 0.01 | 0.02 |
| | | DEV | | | | |

In Table 11, the pH-maintaining effects of a TRIS buffer at a pH value of 7 stored at 40° C. was examined.

TABLE 11
pH of retinol cream comprised of TRIS buffer pH =
7 stored at 40° C. for 12 weeks

| | Buffer | | pH | | | |
|---|---|---|---|---|---|---|
| Experiment # | (wt %) | Trial | Initial | 4 w 40° C. | 8 w 40° C. | 12 w 40° C. |
| 1 | 0.33 | 1 | 6.98 | 6.81 | 7.06 | 7.16 |
| | | 2 | 6.97 | 6.58 | 7.08 | 7.00 |
| | | 3 | 6.94 | 6.73 | 7.12 | 7.17 |
| | | 4 | 6.99 | 6.83 | 7.12 | 7.21 |
| | | AVG | 6.97 | 6.74 | 7.10 | 7.14 |
| | | STD | 0.02 | 0.11 | 0.03 | 0.09 |
| | | DEV | | | | |
| 2 | 0.66 | 1 | 7.12 | 7.15 | 7.07 | 7.26 |
| | | 2 | 7.12 | 7.03 | 7.11 | 7.19 |
| | | 3 | 7.07 | 7.09 | 7.14 | 7.34 |
| | | 4 | 7.03 | 7.02 | 7.15 | 7.28 |
| | | AVG | 7.11 | 7.07 | 7.11 | 7.27 |
| | | STD | 0.03 | 0.06 | 0.03 | 0.06 |
| | | DEV | | | | |
| 3 | 1.00 | 1 | 7.10 | 7.06 | 7.16 | 7.19 |
| | | 2 | 7.12 | 7.12 | 7.13 | 7.16 |
| | | 3 | 7.05 | 7.10 | 7.16 | 7.18 |
| | | 4 | 7.04 | 7.09 | 7.17 | 7.19 |
| | | AVG | 7.08 | 7.09 | 7.16 | 7.18 |
| | | STD | 0.04 | 0.03 | 0.02 | 0.01 |
| | | DEV | | | | |

In Table 12, the pH-maintaining effects of a TRIS buffer at a pH value of 7 stored at 25° C. was examined.

TABLE 12
pH of retinol cream comprised of TRIS buffer pH = 7 stored at 25° C.

| | Buffer | | pH | | | |
|---|---|---|---|---|---|---|
| Experiment # | (wt %) | Trial | Initial | 4 w 25° C. | 8 w 25° C. | 12 w 25° C. |
| 1 | 0.33 | 1 | 6.98 | 6.84 | 7.07 | 6.93 |
| | | 2 | 6.97 | 6.84 | 7.06 | 7.00 |
| | | 3 | 6.94 | 6.88 | 7.09 | 6.95 |
| | | 4 | 6.99 | 6.93 | 6.90 | 6.96 |
| | | AVG | 6.97 | 6.87 | 7.03 | 6.96 |
| | | STD | 0.02 | 0.04 | 0.09 | 0.03 |
| | | DEV | | | | |
| 2 | 0.66 | 1 | 7.12 | 7.07 | 7.04 | 7.23 |
| | | 2 | 7.12 | 7.09 | 7.20 | 7.19 |
| | | 3 | 7.07 | 7.11 | 7.17 | 7.13 |
| | | 4 | 7.03 | 7.14 | 7.13 | 7.21 |
| | | AVG | 7.11 | 7.10 | 7.14 | 7.19 |
| | | STD | 0.03 | 0.03 | 0.07 | 0.04 |
| | | DEV | | | | |
| 3 | 1.00 | 1 | 7.10 | 7.10 | 7.01 | 6.95 |
| | | 2 | 7.12 | 7.03 | 7.02 | 6.99 |
| | | 3 | 7.05 | 7.02 | 7.02 | 7.11 |
| | | 4 | 7.04 | 7.07 | 7.00 | 7.13 |
| | | AVG | 7.08 | 7.06 | 7.01 | 7.05 |
| | | STD | 0.04 | 0.04 | 0.01 | 0.09 |
| | | DEV | | | | |

### Example 5

### (Clinical Study)

A controlled, randomized clinical study was conducted to evaluate the clinical effects of 0.3% retinol on facial skin over a 3-month period. The study was conducted on two groups of female subjects (N=29 for each group) aged 38-69. Subjects were applied a topical composition comprising 0.3% retinol to entire face either nightly or every other night 5-10 minutes after cleansing face. A baseline skin evaluation was taken prior to commencement of study, and subjects' facial skin was examined at 1, 2, 4, 8, and 12 weeks. Facial skin was examined for changes in fine lines, deep lines, firmness, wrinkles, skin tone, mottled hyperpigmentation, radiance, and texture/smoothness, and scores (10-point scale) were assigned to changes in these characteristics, with a score of 10 representing the largest magnitude of change, as compared to a baseline score.

TABLE 12
Percent Improvement Over Baseline
Every Other Night

| | 1 week | 2 weeks | 4 weeks | 8 weeks | 12 weeks |
|---|---|---|---|---|---|
| Deep lines | NS | NS | 20% | 32% | 43% |
| Fine lines | NS | NS | 30% | 31% | 36% |
| Firmness | 24% | 14% | 24% | NS | 28% |
| Mottled | NS | 14% | 13% | 25% | 36% |
| Hyperpigmentation | | | | | |
| Radiance | NS | 15% | 13% | 21% | 24% |
| Texture/Smoothness | 16% | NS | NS | 29% | 52% |
| Uneven skin tone | 21% | 17% | 21% | 27% | 40% |
| Wrinkles | NS | 8% | 17% | 18% | 28% |

NS = no significant change

TABLE 13
Percent Improvement Over Baseline
Every Night

| | 1 week | 2 weeks | 4 weeks | 8 weeks | 12 weeks |
|---|---|---|---|---|---|
| Deep lines | 13% | 19% | 22% | 34% | 44% |
| Fine lines | 19% | 19% | 24% | 32% | 39% |
| Firmness | 21% | 15% | 14% | 15% | 27% |
| Mottled | 8% | 10% | 19% | 28% | 40% |
| Hyperpigmentation | | | | | |
| Radiance | 15% | 12% | 13% | 20% | 27% |
| Texture/Smoothness | NS | NS | NS | 30% | 55% |
| Uneven skin tone | 14% | 14% | 19% | 27% | 37% |
| Wrinkles | NS | 13% | 16% | 20% | 29% |

TABLE 14
Percent Of Panelists That Improved
Every Other Night

| | 1 week | 2 weeks | 4 weeks | 8 weeks | 12 weeks |
|---|---|---|---|---|---|
| Deep lines | 38% | 48% | 62% | 72% | 86% |
| Fine lines | 59% | 66% | 79% | 83% | 93% |
| Firmness | 62% | 41% | 59% | 38% | 62% |
| Mottled | 21% | 59% | 48% | 86% | 100% |
| Hyperpigmentation | | | | | |
| Radiance | 41% | 48% | 52% | 62% | 59% |
| Texture/Smoothness | 62% | 48% | 41% | 79% | 93% |
| Uneven skin tone | 79% | 69% | 79% | 83% | 93% |
| Wrinkles | 24% | 34% | 62% | 62% | 83% |

TABLE 15
Percent Of Panelists That Improved
Every Night

| | 1 week | 2 weeks | 4 weeks | 8 weeks | 12 weeks |
|---|---|---|---|---|---|
| Deep lines | 41% | 59% | 59% | 62% | 86% |
| Fine lines | 59% | 59% | 69% | 79% | 90% |
| Firmness | 55% | 41% | 45% | 41% | 66% |
| Mottled | 31% | 38% | 66% | 86% | 100% |
| Hyperpigmentation | | | | | |
| Radiance | 55% | 52% | 59% | 69% | 72% |
| Texture/Smoothness | 48% | 28% | 48% | 83% | 100% |
| Uneven skin tone | 62% | 52% | 79% | 90% | 97% |
| Wrinkles | 28% | 55% | 66% | 66% | 90% |

The clinical study results show that nightly application, or application every other night, of a skincare composition comprising 0.3% retinol led to improvements in subjects' facial skin wrinkles, fine lines, deep lines, firmness, mottled hyperpigmentation, radiance, texture, smoothness, and uneven skin tone. In subjects who applied the skincare composition every other night, improvements in firmness, texture/smoothness, and uneven skin tone were realized within 1 week of use. Subjects who applied the skincare composition every night saw improvements in fine lines, deep lines, firmness, mottled hyperpigmentation, radiance, and uneven skin tone within the first week of use. 100% of study subjects experienced improvements in mottled hyperpigmentation and skin texture/smoothness at the 12-week study endpoint.

86% of subjects who applied the skincare composition every night experienced an improvement in facial skin deep lines at the 12-week study endpoint. 90% of these subjects experienced an improvement in facial skin fine lines. 66% of the subjects experienced an improvement in facial skin firmness. 72% of the subjects experienced an improvement in facial skin radiance. 97% of the subjects experienced an improvement in facial skin tone unevenness. 90% of the subjects experienced an improvement in facial skin wrinkles.

86% of subjects who applied the skincare composition every other night experienced an improvement in facial skin deep lines at the 12-week study endpoint. 93% of these subjects experienced an improvement in facial skin fine lines. 62% of the subjects experienced an improvement in facial skin firmness. 59% of the subjects experienced an improvement in facial skin radiance. 93% of the subjects experienced an improvement in facial skin tone unevenness. 83% of the subjects experienced an improvement in facial skin wrinkles.

Subjects who applied the skincare composition every night experienced an average improvement in facial skin deep lines of 44%, and an improvement in fine lines of 39%. Facial skin firmness increased by 27% and mottled hyperpigmentation was reduced by 40%. Facial skin radiance improved by 27% and skin texture/smoothness improved 55%. Uneven skin tones were reduced 37% and overall facial skin wrinkling was reduced 29%.

Similar results were observed for subjects who applied the skincare composition every night. An average improvement in facial skin deep lines of 45% was observed, and an improvement in fine lines of 36% was observed. %. Facial skin firmness increased by 28% and mottled hyperpigmentation was reduced by 36%. Facial skin radiance improved by 24% and skin texture/smoothness improved 52%. Uneven skin tones were reduced by 40% and overall facial skin wrinkling was reduced 28%. The improvements for both nightly and application every other night application were time-dependent, as the improvements in these attributes increased over the course of the 12-week study. Some attribute improvements were not realized until after three weeks of use, however, all attributes that were examined saw improvements at the 12-week study endpoint.

The results of the clinical study described above show that improvements were realized in all examined categories. Rough skin felt smoother and lax skin became firmer. 100% of subjects' skin texture became smoother. Skin smoothness doubled. 90% of subjects' fine lines and wrinkles were less visible. 86% of subjects showed a reduction in deep lines, and deep lines were improved by an average of 44%. 100% of subjects has less blotchiness, and skin tone evenness increased by 37%. Rough skin felt smoother and lax skin became firmer. 100% of subjects' skin texture became smoother. Skin smoothness doubled. 90% of subjects' fine lines and wrinkles were less visible. 86% of subjects showed a reduction in deep lines, and deep lines were improved by an average of 44%. 100% of subjects had less skin blotchiness, and skin tone evenness increased by 37%.

Additionally, the use of a skincare composition comprising 0.3% retinol led to improvements in fine lines, deep lines, and mottled hyperpigmentation after only one week of use (data not shown). These results demonstrate that the compositions disclosed herein provide improvements in skin attributes that are typically associated with ageing, and provide real-world skin-renovating abilities.

### Example 6

### (Additional Assays)

Assays that can be used to determine the efficacy of any one of the ingredients or any combination of ingredients or compositions having said combination of ingredients disclosed throughout the specification and claims can be determined by methods known to those of ordinary skill in the art. The following are non-limiting assays that can be used in the context of the present invention. It should be recognized that other testing procedures can be used, including, for example, objective and subjective procedures.

B16 Pigmentation Assay: Melanogenesis is the process by which melanocytes produce melanin, a naturally produced pigment that imparts color to skin, hair, and eyes. Inhibiting melanogenesis is beneficial to prevent skin darkening and lighten dark spots associated with aging. This bioassay utilizes B16-F1 melanocytes (ATCC), an immortalized mouse melanoma cell line, to analyze the effect of compounds on melanogenesis. The endpoint of this assay is a spectrophotometric measurement of melanin production and cellular viability. B16-F1 melanocytes, can be cultivated in standard DMEM growth medium with 10% fetal bovine serum (MEDIATECH) at 37° C. in 10% CO₂ and then treated with any one of the active ingredients, combination of ingredients, or compositions having said combinations disclosed in the specification for 6 days. Following incubation, melanin secretion is measured by absorbance at 405 nm and cellular viability is quantified.

Collagen Stimulation Assay: Collagen is an extracellular matrix protein critical for skin structure. Increased synthesis of collagen helps improve skin firmness and elasticity. This bioassay can be used to examine the effect of any one of the active ingredients, combination of ingredients, or compositions having said combinations disclosed in the specification on the production of procollagen peptide (a precursor to collagen) by human epidermal fibroblasts. The endpoint of this assay is a spectrophotometric measurement that reflects the presence of procollagen peptide and cellular viability. The assay employs the quantitative sandwich enzyme immunoassay technique whereby a monoclonal antibody specific for procollagen peptide has been pre-coated onto a microplate. Standards and samples can be pipetted into the wells and any procollagen peptide present is bound by the immobilized antibody. After washing away any unbound substances, an enzyme-linked polyclonal antibody specific for procollagen peptide can be added to the wells. Following a wash to remove any unbound antibody-enzyme reagent, a substrate solution can be added to the wells and color develops in proportion to the amount of procollagen peptide bound in the initial step using a microplate reader for detection at 450 nm. The color development can be stopped and the intensity of the color can be measured.

For generation of samples and controls, subconfluent normal human adult epidermal fibroblasts (Cascade Biologics) cultivated in standard DMEM growth medium with 10% fetal bovine serum (MEDIATECH) at 37° C. in 10% CO₂, can be treated with each of the combination of ingredients or compositions having said combinations disclosed in the specification for 3 days. Following incubation, cell culture medium can be collected and the amount of procollagen peptide secretion quantified using a sandwich enzyme linked immuno-sorbant assay (ELISA) from TAKARA (#MK101).

Elastin Stimulation Assay: Elastin is a connective tissue protein that helps skin resume shape after stretching or contracting. Elastin is also an important load-bearing protein used in places where mechanical energy is required to be stored. Elastin is made by linking many soluble tropoclastin protein molecules, in a reaction catalyzed by lysyl oxidase. Elastin secretion and elastin fibers can be monitored in cultured human fibroblasts by staining of cultured human fibroblasts using immunofluorescent antibodies directed against elastin.

Laminin and Fibronectin Stimulation Assay: Laminin and fibronectin are major proteins in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Laminin and fibronectin are two structural glycoproteins located in the DEJ. Considered the glue that holds the cells together, laminin and fibronectin are secreted by dermal fibroblasts to help facilitate intra- and inter-cellular adhesion of the epidermal calls to the DEJ. Laminin and fibronectin secretion can be monitored by quantifying laminin and fibronectin in cell supernatants of cultured human fibroblasts treated for 3 days with culture medium with or without 1.0% final concentration of the test ingredient(s). Following incubation, laminin and fibronectin content can be measured using immunofluorescent antibodies directed against laminin and antibodies directed against fibronectin in an enzyme linked immunosorbent assay (ELISA). Measurements are normalized for cellular metabolic activity, as determined by bioconversion of 3-(4,5-dimethylthiazol-2-yl)-5-(3-carboxymethoxyphenyl)-2-(4-sulfophenyl)-2H-tetrazolium (MTS).

Tumor Necrosis Factor Alpha (TNF-α) Assay: The prototype ligand of the TNF superfamily, TNF-α, is a pleiotropic cytokine that plays a central role in inflammation. Increase in its expression is associated with an up regulation in pro-inflammatory activity. This bioassay can be used to analyze the effect of any one of the active ingredients, combination of ingredients, or compositions having said combinations disclosed in the specification on the production of TNF-α by human epidermal keratinocytes. The endpoint of this assay can be a spectrophotometric measurement that reflects the presence of TNF-α and cellular viability. The assay employs the quantitative sandwich enzyme immunoassay technique whereby a monoclonal antibody specific for TNF-α has been pre-coated onto a microplate. Standards and samples can be pipetted into the wells and any TNF-α present is bound by the immobilized antibody. After washing away any unbound substances, an enzyme-linked polyclonal antibody specific for TNF-α can be added to the wells. Following a wash to remove any unbound antibody-enzyme reagent, a substrate solution can be added to the wells and color develops in proportion to the amount of TNF-α bound in the initial step using a microplate reader for detection at 450 nm. The color development can be stopped and the intensity of the color can be measured. Subconfluent normal human adult keratinocytes (Cascade Biologics) cultivated in EPILIFE™ standard growth medium (Cascade Biologics) at 37° C. in 5% CO₂, can be treated with phorbol 12-myristate 13-acetate (PMA, 10 ng/ml, SIGMA CHEMICAL, #P1585-1 MG) and any one of the active ingredients, combination of ingredients, or compositions having said combinations disclosed in the specification for 6 hours. PMA has been shown to cause a dramatic increase in TNF-α secretion which peaks at 6 hours after treatment. Following incubation, cell culture medium can be collected and the amount of TNF-α secretion quantified using a sandwich enzyme linked immuno-sorbant assay (ELISA) from R&D Systems (#DTA00C).

Antioxidant (AO) Assay: An in vitro bioassay that measures the total anti-oxidant capacity of any one of the ingredients, combination of ingredients, or compositions having said combinations disclosed in the specification. The assay relies on the ability of antioxidants in the sample to inhibit the oxidation of ABTS® (2,2′-azino-di-[3-ethylbenzthiazoline sulphonate]) to ABTS®.+by metmyoglobin. The antioxidant system of living organisms includes enzymes such as superoxide dismutase, catalase, and glutathione peroxidase; macromolecules such as albumin, ceruloplasmin, and ferritin; and an array of small molecules, including ascorbic acid, α-tocopherol, β-carotene, reduced glutathione, uric acid, and bilirubin. The sum of endogenous and food-derived antioxidants represents the total antioxidant activity of the extracellular fluid. Cooperation of all the different antioxidants provides greater protection against attack by reactive oxygen or nitrogen radicals, than any single compound alone. Thus, the overall antioxidant capacity may give more relevant biological information compared to that obtained by the measurement of individual components, as it considers the cumulative effect of all antioxidants present in plasma and body fluids. The capacity of the antioxidants in the sample to prevent ABTS® oxidation is compared with that of Trolox, a water-soluble tocopherol analogue, and is quantified as molar Trolox equivalents. Anti-Oxidant capacity kit #709001 from CAYMAN CHEMICAL (Ann Arbor, Michigan USA) can be used as an in vitro bioassay to measure the total anti-oxidant capacity of each of any one of the active ingredients, combination of ingredients, or compositions having said combinations disclosed in the specification. The protocol can be followed according to manufacturer recommendations.

ORAC Assay: Oxygen Radical Absorption (or Absorbance) Capacity (ORAC) of any one of the active ingredients, combination of ingredients, or compositions having said combinations disclosed in the specification can also be assayed by measuring the antioxidant activity of such ingredients or compositions. Antioxidant activity indicates a capability to reduce oxidizing agents (oxidants). This assay quantifies the degree and length of time it takes to inhibit the action of an oxidizing agent, such as oxygen radicals, that are known to cause damage to cells (e.g., skin cells). The ORAC value of any one of the active ingredients, combination of ingredients, or compositions having said combinations disclosed in the specification can be determined by methods known to those of ordinary skill in the art (see U.S. Publication Nos. 2004/0109905 and 2005/0163880; and commercially available kits such as Zen-Bio ORAC Anti-oxidant Assay kit (#AOX-2)). The Zen-Bio ORAC Anti-oxidant Assay kit measures the loss of fluorescein fluorescence over time due to the peroxyl-radical formation by the breakdown of AAPH (2,2′-axobis-2-methyl propanimidamide, dihydrochloride). Trolox, a water soluble vitamin E analog, serves as positive control inhibition fluorescein decay in a dose dependent manner.

Mushroom tyrosinase activity assay: In mammalian cells, tyrosinase catalyzes two steps in the multi-step biosynthesis of melanin pigments from tyrosine (and from the polymerization of dopachrome). Tyrosinase is localized in melanocytes and produces melanin (aromatic quinone compounds) that imparts color to skin, hair, and eyes. Purified mushroom tyrosinase (from SIGMA) can be incubated with its substrate L-Dopa (from FISHER) in the presence or absence of each of the active ingredients, any one of the combination of ingredients, or compositions having said combinations disclosed in the specification. Pigment formation can be evaluated by colorimetric plate reading at 490 nm. The percent inhibition of mushroom tyrosinase activity can be calculated compared to non-treated controls to determine the ability of test ingredients or combinations thereof to inhibit the activity of purified enzyme. Test extract inhibition was compared with that of kojic acid (SIGMA).

Matrix Metalloproteinase 3 and 9 Enzyme Activity (MMP3; MMP9) Assay: An in vitro matrix metalloprotease (MMP) inhibition assay. MMPs are extracellular proteases that play a role in many normal and disease states by virtue of their broad substrate specificity. MMP3 substrates include collagens, fibronectins, and laminin; while MMP9 substrates include collagen VII, fibronectins and laminin. Using Colorimetric Drug Discovery kits from BioMol International for MMP3 (AK-400) and MMP-9 (AK-410), this assay is designed to measure protease activity of MMPs using a thiopeptide as a chromogenic substrate (Ac-PLG-[2-mercapto-4-methyl-pentanoyl]-LG-OC2H5)5,6. The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, which can be detected by its absorbance at 412 nm (ε=13,600 M-1cm⁻¹ at pH 6.0 and above 7). The active ingredients, any one of the combination of ingredients, or compositions having said combinations disclosed in the specification can be assayed.

Matrix Metalloproteinase 1 Enzyme Activity (MMP1) Assay: An in vitro matrix metalloprotease (MMP) inhibition assay. MMPs are extracellular proteases that play a role in many normal and disease states by virtue of their broad substrate specificity. MMPI substrates include collagen IV. The MOLECULAR PROBES ENZ/CHEK GELATINASE/COLLAGENASE ASSAY kit (#E12055) utilizes a fluorogenic gelatin substrate to detect MMPI protease activity. Upon proteolytic cleavage, bright green fluorescence is revealed and may be monitored using a fluorescent microplate reader to measure enzymatic activity.

The ENZ/CHEK GELATINASE/COLLAGENASE ASSAY kit (#E12055) from Invitrogen is designed as an in vitro assay to measure MMPI enzymatic activity. The active ingredients, any one of the combination of ingredients, or compositions having said combinations disclosed in the specification can be assayed. The assay relies upon the ability of purified MMPI enzyme to degrade a fluorogenic gelatin substrate. Once the substrate is specifically cleaved by MMPI bright green fluorescence is revealed and may be monitored using a fluorescent microplate reader. Test materials are incubated in the presence or absence of the purified enzyme and substrate to determine their protease inhibitor capacity.

Cyclooxygenase (COX) Assay: An in vitro cyclooxygenase-1 and -2 (COX-1,-2) inhibition assay. COX is a bifunctional enzyme exhibiting both cyclooxygenase and peroxidase activities. The cyclooxygenase activity converts arachidonic acid to a hydroperoxy endoperoxide (Prostaglandin G2; PGG2) and the peroxidase component reduces the endoperoxide (Prostaglandin H2; PGH2) to the corresponding alcohol, the precursor of prostaglandins, thromboxanes, and prostacyclins. This COX Inhibitor screening assay measures the peroxidase component of cyclooxygenases. The peroxidase activity is assayed colorimetrically by monitoring the appearance of oxidized N,N,N′,N′-tetramethyl-p-phenylenediamine (TMPD). This inhibitor screening assay includes both COX-1 and COX-2 enzymes in order to screen isozyme-specific inhibitors. The Colormetric COX (ovine) Inhibitor screening assay (#760111, CAYMAN CHEMICAL) can be used to analyze the effects of each of the active ingredients, any one of the combination of ingredients, or compositions having said combinations disclosed in the specification on the activity of purified cyclooxygenase enzyme (COX-1 or COX-2). According to manufacturer instructions, purified enzyme, heme and test extracts can be mixed in assay buffer and incubated with shaking for 15 min at room temperature. Following incubation, arachidonic acid and colorimetric substrate can be added to initiate the reaction. Color progression can be evaluated by colorimetric plate reading at 590 nm. The percent inhibition of COX-1 or COX-2 activity can be calculated compared to non-treated controls to determine the ability of test extracts to inhibit the activity of purified enzyme.

Lipoxygenase (LO) Assay: An in vitro lipoxygenase (LO) inhibition assay. LOs are non-heme iron-containing dioxygenases that catalyze the addition of molecular oxygen to fatty acids. Linoleate and arachidonate are the main substrates for LOs in plants and animals. Arachadonic acid may then be converted to hydroxyeicosotrienenoic (HETE) acid derivatives, that are subsequently converted to leukotrienes, potent inflammatory mediators. This assay provides an accurate and convenient method for screening lipoxygenase inhibitors by measuring the hydroperoxides generated from the incubation of a lipoxygenase (5-, 12-, or 15-LO) with arachidonic acid. The Colorimetric LO Inhibitor screening kit (#760700, CAYMAN CHEMICAL) can be used to determine the ability of each of the active ingredients, any one of the combination of ingredients, or compositions having said combinations disclosed in the specification to inhibit enzyme activity. Purified 15-lipoxygenase and test ingredients can be mixed in assay buffer and incubated with shaking for 10 min at room temperature. Following incubation, arachidonic acid can be added to initiate the reaction and the mixtures can be incubated for an additional 10 min at room temperature. Colorimetric substrate can be added to terminate catalysis and color progression can be evaluated by fluorescence plate reading at 490 nm. The percent inhibition of lipoxyganse activity can be calculated compared to non-treated controls to determine the ability of each of the active ingredients, any one of the combination of ingredients, or compositions having said combinations disclosed in the specification to inhibit the activity of purified enzyme.

Elastase Assay: ENZCHEK® Elastase Assay (Kit #E-12056) from MOLECULAR PROBES (Eugene, Oregon USA) can be used as an in vitro enzyme inhibition assay for measuring inhibition of elastase activity for each of the active ingredients, any one of the combination of ingredients, or compositions having said combinations disclosed in the specification. The ENZCHEK kit contains soluble bovine neck ligament elastin that can be labeled with dye such that the conjugate's fluorescence can be quenched. The non-fluorescent substrate can be digested by elastase or other proteases to yield highly fluorescent fragments. The resulting increase in fluorescence can be monitored with a fluorescence microplate reader. Digestion products from the elastin substrate have absorption maxima at ˜505 nm and fluorescence emission maxima at ˜515 nm. The peptide, N-methoxysuccinyl-Ala-Ala-Pro-Val-chloromethyl ketone, can be used as a selective, collective inhibitor of elastase when utilizing the ENZCHEK ELASTASE ASSAY KIT for screening for elastase inhibitors.

Production of Ceramides: Ceramides in cell or tissue samples can be labeled with a mouse monoclonal antibody anti-ceramide (ENZO LIFE SCIENCE, ref ALX-804-196 clone MID15B4) diluted to 1/50 for 2 hours at room temperature with an amplifier system biotin/streptavidin. Video microscope observation can be performed to view ceramides (pink stain).

Oil Control Assay: An assay to measure reduction of sebum secretion from sebaceous glands and/or reduction of sebum production from sebaceous glands can be assayed by using standard techniques known to those having ordinary skill in the art. In one instance, the forehead can be used. Each of the active ingredients, any one of the combination of ingredients, or compositions having said combinations disclosed in the specification can be applied to one portion of the forehead once or twice daily for a set period of days (e.g., 1, 2, 3, 4, 5, 6, 7, 8, 9, 10, 11, 12, 13, 14, or more days), while another portion of the forehead is not treated with the composition. After the set period of days expires, then sebum secretion can be assayed by application of fine blotting paper to the treated and untreated forehead skin. This is done by first removing any sebum from the treated and untreated areas with moist and dry cloths. Blotting paper can then be applied to the treated and untreated areas of the forehead, and an elastic band can be placed around the forehead to gently press the blotting paper onto the skin. After 2 hours the blotting papers can be removed, allowed to dry and then transilluminated. Darker blotting paper correlates with more sebum secretion (or lighter blotting paper correlates with reduced sebum secretion.

Erythema Assay: An assay to measure the reduction of skin redness can be evaluated using a MINOLTA chroma meter. Skin erythema may be induced by applying a 0.2% solution of sodium dodecyl sulfate on the forearm of a subject. The area is protected by an occlusive patch for 24 hrs. After 24 hrs, the patch is removed and the irritation-induced redness can be assessed using the a* values of the MINOLTA chroma meter. The a* value measures changes in skin color in the red region. Immediately after reading, the area is treated with the active ingredients, any one of the combination of ingredients, or compositions having said combinations disclosed in the specification. Repeat measurements can be taken at regular intervals to determine the formula's ability to reduce redness and irritation.

Skin Moisture/Hydration Assay: Skin moisture/hydration benefits can be measured by using impedance measurements with the Nova Dermal Phase Meter. The impedance meter measures changes in skin moisture content. The outer layer of the skin has distinct electrical properties. When skin is dry it conducts electricity very poorly. As it becomes more hydrated increasing conductivity results. Consequently, changes in skin impedance (related to conductivity) can be used to assess changes in skin hydration. The unit can be calibrated according to instrument instructions for each testing day. A notation of temperature and relative humidity can also be made. Subjects can be evaluated as follows: prior to measurement they can equilibrate in a room with defined humidity (e.g., 30-50%) and temperature (e.g., 68-72° C.). Three separate impedance readings can be taken on each side of the face, recorded, and averaged. The T5 setting can be used on the impedance meter which averages the impedance values of every five seconds application to the face. Changes can be reported with statistical variance and significance. Each of the active ingredients, any one of the combination of ingredients, or compositions having said combinations disclosed in the specification can be assayed according to this process.

Skin Clarity and Reduction in Freckles and Age Spots Assay: Skin clarity and the reduction in freckles and age spots can be evaluated using a Minolta Chromometer. Changes in skin color can be assessed to determine irritation potential due to product treatment using the a* values of the Minolta Chroma Meter. The a* value measures changes in skin color in the red region. This is used to determine whether each of the active ingredients, any one of the combination of ingredients, or compositions having said combinations disclosed in the specification is inducing irritation. The measurements can be made on each side of the face and averaged, as left and right facial values. Skin clarity can also be measured using the Minolta Meter. The measurement is a combination of the a*, b, and L values of the Minolta Meter and is related to skin brightness, and correlates well with skin smoothness and hydration. Skin reading is taken as above. In one non-limiting aspect, skin clarity can be described as L/C where C is chroma and is defined as (a²+b²)^{1/2}.

Skin Dryness, Surface Fine Lines, Skin Smoothness, and Skin Tone Assay: Skin dryness, surface fine lines, skin smoothness, and skin tone can be evaluated with clinical grading techniques. For example, clinical grading of skin dryness can be determined by a five point standard Kligman Scale: (0) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations can be made independently by two clinicians and averaged.

Clinical Grading of Skin Tone Assay: Clinical grading of skin tone can be performed via a ten point analog numerical scale: (10) even skin of uniform, pinkish brown color. No dark, erythremic, or scaly patches upon examination with a hand held magnifying lens. Microtexture of the skin very uniform upon touch; (7) even skin tone observed without magnification. No scaly areas, but slight discolorations either due to pigmentation or erythema. No discolorations more than 1 cm in diameter; (4) both skin discoloration and uneven texture easily noticeable. Slight scaliness. Skin rough to the touch in some areas; and (1) uneven skin coloration and texture. Numerous areas of scaliness and discoloration, either hypopigmented, erythremic or dark spots. Large areas of uneven color more than 1 cm in diameter. Evaluations were made independently by two clinicians and averaged.

Clinical Grading of Skin Smoothness Assay: Clinical grading of skin smoothness can be analyzed via a ten point analog numerical scale: (10) smooth, skin is moist and glistening, no resistance upon dragging finger across surface; (7) somewhat smooth, slight resistance; (4) rough, visibly altered, friction upon rubbing; and (1) rough, flaky, uneven surface. Evaluations were made independently by two clinicians and averaged.

Skin Smoothness and Wrinkle Reduction Assay With Methods Disclosed in Packman et al. (1978): Skin smoothness and wrinkle reduction can also be assessed visually by using the methods disclosed in Packman et al. (1978). For example, at each subject visit, the depth, shallowness and the total number of superficial facial lines (SFLs) of each subject can be carefully scored and recorded. A numerical score was obtained by multiplying a number factor times a depth/width/length factor. Scores are obtained for the eye area and mouth area (left and right sides) and added together as the total wrinkle score.

Skin Firmness Assay with a Hargens Ballistometer: Skin firmness can be measured using a Hargens ballistometer, a device that evaluates the elasticity and firmness of the skin by dropping a small body onto the skin and recording its first two rebound peaks. The ballistometry is a small lightweight probe with a relatively blunt tip (4 square mm-contact area) was used. The probe penetrates slightly into the skin and results in measurements that are dependent upon the properties of the outer layers of the skin, including the stratum corneum and outer epidermis and some of the dermal layers.

Skin Softness/Suppleness Assay with a Gas Bearing Electrodynamometer: Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the check area by attaching the probe to the skin surface with double-stick tape. A force of approximately 3.5 gm can be applied parallel to the skin surface and the skin displacement is accurately measured. Skin suppleness can then be calculated and is expressed as DSR (Dynamic Spring Rate in gm/mm).

Appearance of Lines and Wrinkles Assay with Replicas: The appearance of lines and wrinkles on the skin can be evaluated using replicas, which is the impression of the skin's surface. Silicone rubber like material can be used. The replica can be analyzed by image analysis. Changes in the visibility of lines and wrinkles can be objectively quantified via the taking of silicon replicas form the subjects' face and analyzing the replicas image using a computer image analysis system. Replicas can be taken from the eye area and the neck area, and photographed with a digital camera using a low angle incidence lighting. The digital images can be analyzed with an image processing program and are of the replicas covered by wrinkles or fine lines was determined.

Surface Contour of the Skin Assay with a Profilometer/Stylus Method: The surface contour of the skin can be measured by using the Profilometer/Stylus method. This includes either shining a light or dragging a stylus across the replica surface. The vertical displacement of the stylus can be fed into a computer via a distance transducer, and after scanning a fixed length of replica a cross-sectional analysis of skin profile can be generated as a two-dimensional curve. This scan can be repeated any number of times along a fix axis to generate a simulated 3-D picture of the skin. Ten random sections of the replicas using the stylus technique can be obtained and combined to generate average values. The values of interest include Ra which is the arithmetic mean of all roughness (height) values computed by integrating the profile height relative to the mean profile height. Rt which is the maximum vertical distance between the highest peak and lowest trough, and Rz which is the mean peak amplitude minus the mean peak height. Values are given as a calibrated value in mm. Equipment should be standardized prior to each use by scanning metal standards of know values. Ra Value can be computed by the following equation: Rₐ=Standardize roughness; lₘ=the traverse (scan) length; and y=the absolute value of the location of the profile relative to the mean profile height (x-axis).

MELANODERM™ Assay: In other non-limiting aspects, the efficacy of each of the active ingredients, any one of the combination of ingredients, or compositions having said combinations disclosed in the specification can be evaluated by using a skin analog, such as, for example, MELANODERM™. Melanocytes, one of the cells in the skin analog, stain positively when exposed to L-dihydroxyphenyl alanine (L-DOPA), a precursor of melanin. The skin analog, MELANODERM™, can be treated with a variety of bases containing each of the active ingredients, any one of the combination of ingredients, or compositions having said combinations disclosed in the specification or with the base alone as a control. Alternatively, an untreated sample of the skin analog can be used as a control.

Production of Filaggrin: Changes in the production of filaggrin in keratinocytes due to each of the active ingredients, any one of the combination of ingredients, or compositions having said combinations disclosed in the specification can be measured. Filaggrin is the precursor to Natural Moisturizing Factor (NMF) in the skin. Increased NMF increases the moisture content of the skin. Filaggrin production in treated and non-treated keratinocytes can be determined using a bioassay that analyzes filaggrin concentration in keratinocyte cell lysates. A non-limiting example of a bioassay that can be used to quantify filaggrin production is the PROTEINSIMPLE® SIMON™ western blotting protocol. For each sample, normal human epidermal keratinocytes (NHEK) are grown in EPI-200-MATTEK EPILIFE® growth media with calcium from Life Technologies (M-EP-500-CA). NHEK are incubated in growth medium overnight at 37° C. in 5% CO₂ prior to treatment. NHEK are then incubated in growth medium with 1% test compound/extract or no compound/extract (negative control) for 24 to 36 hours. The NHEK can then be washed, collected, and stored on ice or colder until lysed on ice using a lysis buffer and sonication. The protein concentrations of the samples can be determined and used to normalize the samples. The lysates can be stored at −80° C. until use in the quantification assay.

The PROTEINSIMPLE® SIMON™ western blotting bioassay assay employs a quantitative western blotting immunoassay technique using an antibody specific for filaggrin to quantitatively detect filaggrin in the test samples. Cell samples are lysed and normalized for protein concentration. Normalized samples and molecular weight standards can then be loaded and ran on a denatured protein separation gel using capillary electrophoresis. The proteins in the gel are immobilized and immunoprobed using a primary antibody specific for filaggrin. The immobilized proteins can then be immunoprobed with an enzyme-linked detection antibody that binds the primary antibody. A chemiluminescent substrate solution can then be added to the immobilized proteins to allow chemiluminescent development in proportion to the amount of filaggrin bound in the immobilization. The chemiluminescent development is stopped at a specific time and the intensity of the chemiluminescent signal can be measured and compared to positive and negative controls.

Production of Occludin: Changes in the production of occludin in keratinocytes due to each of the active ingredients, any one of the combination of ingredients, or compositions having said combinations disclosed in the specification can be measured. Occludin is a protein critical to the formulation of tight junctions and the skin's moisture barrier function. A non-limiting example of how occludin production in treated and non-treated keratinocytes can be determined is by the use of a bioassay that analyzes occludin concentration in keratinocyte cell lysates. The bioassay can be performed using PROTEINSIMPLE® SIMON™ western blotting protocol. For the samples, adult human epidermal keratinocytes (HEKa) from Life Technologies (C-005-5C) can be grown at 37° C. and 5% CO₂ for 24 hours in EPILIFE™ growth media with calcium from Life Technologies (M-EP-500-CA) supplemented with Keratinocyte Growth Supplement (HKGS) from Life Technologies (S-101-5). HEKa are then incubated in growth medium with test compound/extract, no compound/extract for negative control, or with 1 mM CaCl₂ for positive control for 24 to 48 hours. The HEKa are then washed, collected, and stored on ice or colder until lysed on ice using a lysis buffer and sonication. The protein concentrations of the samples can be determined and used to normalize the samples. The lysates are stored at −80° C. until use in the bioassay.

The PROTEINSIMPLE® SIMON™ western blotting bioassay assay employs a quantitative western blotting immunoassay technique using an antibody specific for occludin to quantitatively detect occludin in the test samples. Cell samples are lysed and normalized for protein concentration. Normalized samples and molecular weight standards are then loaded and ran on a denatured protein separation gel using capillary electrophoresis. The proteins in the gel are then immobilized and immunoprobed using a primary antibody specific for occludin. The immobilized proteins are immunoprobed with an enzyme-linked detection antibody that binds the primary antibody. A chemiluminescent substrate solution is then added to the immobilized proteins to allow chemiluminescent development in proportion to the amount of occludin bound in the immobilization. The chemiluminescent development can be stopped at a specific time and the intensity of the chemiluminescent signal can be measured and compared to positive and negative controls.

Keratinocyte Monolayer Permeability: Changes in the permeability of a keratinocyte monolayer due to each of the active ingredients, any one of the combination of ingredients, or compositions having said combinations disclosed in the specification can be measured. Keratinocyte monolayer permeability is a measure of skin barrier integrity. Keratinocyte monolayer permeability in treated and non-treated keratinocytes can be determined using, as a non-limiting example, the In Vitro Vascular Permeability assay by MILLIPORE (ECM642). This assay analyzes endothelial cell adsorption, transport, and permeability. Briefly, adult human epidermal keratinocytes from Life Technologies (C-005-5C) can be seeded onto a porous collagen-coated membrane within a collection well. The keratinocytes are then incubated for 24 hours at 37° C. and 5% CO₂ in EPILIFE™ growth media with calcium from LIFE TECHNOLOGIES (M-EP-500-CA) supplemented with Keratinocyte Growth Supplement (HKGS) from LIFE TECHNOLOGIES (S-101-5). This incubation time allows the cells to form a monolayer and occlude the membrane pores. The media is then replaced with fresh media with (test sample) or without (non-treated control) test compounds/extracts and the keratinocytes are incubated for an additional 48 hours at 37° C. and 5% CO₂. To determine permeability of the keratinocyte monolayer after incubation with/without the test compound/extract, the media is replaced with fresh media containing a high molecular weight Fluorescein isothiocyanate (FITC)-Dextran and the keratinocytes are incubated for 4 hours at 37° C. and 5% CO₂. During the 4 hours incubation, FITC can pass through the keratinocytes monolayer and porous membrane into the collection well at a rate proportional to the monolayer's permeability. After the 4 hour incubation, cell viability and the content of FITC in the collection wells can be determined. For the FITC content, the media in the collection well is collected and fluorescence of the media determined at 480 nm (Em) when excited at 520 nm. Percent permeability and percent change in comparison to the non-treated controls can be determined by the following equations: Percent Permeability=((Mean Ex/Em of test sample)/Mean Ex/Em untreated control)*100; Percent Change=Percent Permeability of test sample-Percent Permeability of untreated control.

Production of Hyaluronic Acid: Changes in the production of hyaluronic acid in human dermal fibroblasts due to each of the active ingredients, any one of the combination of ingredients, or compositions having said combinations disclosed in the specification can be measured. HA is a polysaccharide involved in stabilization of the structure of the matrix and is involved in providing turgor pressure to tissue and cells.

As one non-limiting example, HA production in treated and non-treated adult human dermal fibroblasts (HDFa) cells can be determined using the Hyaluronan DuoSet ELISA kit from R&D Systems (DY3614). In this assay, for production of samples, subconfluent HDFa cells from Cascade Biologics (C-13-5C) are incubated at 37° C. and 10% CO₂ in starvation medium (0.15% fetal bovine serum and 1% Penicillin Streptomycin solution in Dulbecco's Modified Eagle Medium) for 72 hours prior to treatment. The cells are then incubated with fresh starvation medium with either test compound, positive control (phorbol 12-myristate 13-acetate from SIGMA-ALDRICH (P1585) and platelet derived growth factor from SIGMA-ALDRICH (P3201)), or no additive for 24 hours. Media is then collected and frozen at −80° C. until use in the ELISA assay.

Briefly, the ELISA assay employs a quantitative sandwich enzyme immunoassay technique whereby a capture antibody specific for HA can be pre-coated onto a microplate. Standards and media from treated and untreated cells are pipetted into the microplate wells to enable any HA present to be bound by the immobilized antibody. After washing away any unbound substances, an enzyme-linked detection antibody specific for HA is added to the wells. Following a wash to remove any unbound antibody-enzyme reagent, a substrate solution is added to the wells to allow color development in proportion to the amount of HA bound in the initial step. The color development is stopped at a specific time and the intensity of the color at 450 nm can be measured using a microplate reader.

As another non-limiting example, human skin explants can be cultured in survival explants medium at 37° C. in a humidified atmosphere supplemented with 5% CO₂. Treatment of the explants can be carried out by topical application of sample product (n=3) on days D0, D2, D3, D6, D8, and D9. The control explants (n=3) receive no treatment except renewal of survival explants medium. Half of the volume of the survival medium can be renewed at days D3, D6, and D8. At D9, three explants of each condition can be taken and cut in half. A half explant is fixed in buffered formalin and the other is frozen at −80° C.

After 48 hours of fixation in ordinary Bouin and 24 hours in formalin, the samples can be dried and soaked in paraffin using an automatic tissue processor Leica TP 1020. Sections of 5 microns can be performed with a microtome (Minot type LEICA RM2125) and mounted on SUPERFROST™ histological slides. Microscopic observations can be performed by optical microscopy, using a LEICA ORTHOPLAN or LEICA DM LB microscope. Images can be taken with an OLYMPUS DP72 camera and CELL\hat{ }D software. General morphology can be examined on paraffin sections stained with Masson's trichrome Goldner variant. The staining of hyaluronic acid can be performed with an anti-hyaluronic acid biotinylated protein (HABP) (SEIKAGAKU ref 400763-1A) diluted to 1/100 for 1 hour at room temperature, with an amplifier system biotin/streptavidin (VECTOR, VECTASTAIN PK-7200).

Inhibition of Hyaluronidase Activity: Changes in the activity of hyaluronidase due to each of the active ingredients, any one of the combination of ingredients, or compositions having said combinations disclosed in the specification can be measured. Hyaluronidase is an enzyme that degrades HA. HA is a polysaccharide involved in stabilization of the structure of the matrix and is involved in providing turgor pressure to tissue and cells. As one non-limiting example, hyaluronidase activity can be determined using an in vitro protocol modified from SIGMA-ALDRICH protocol #EC 3.2.1.35. Briefly, hyaluronidase type 1-S from SIGMA-ALDRICH (H3506) is added to microplate reaction wells containing test compound or controls. Tannic acid can be used as a positive control inhibitor, no test compound can be added for the control enzyme, and wells with test compound or positive control but without hyaluronidase can be used as a background negative control. The wells are incubated at 37° C. for 10 minutes before addition of substrate (HA). Substrate is added and the reactions incubated at 37° C. for 45 minutes. A portion of each reaction solution is then transferred to and gently mixed in a solution of sodium acetate and acetic acid pH 3.75 to stop that portion of the reaction (stopped wells). The stopped wells and the reaction wells should both contain the same volume of solution after addition of the portion of the reaction solution to the stopped wells. Both the reaction wells and the stopped wells are incubated for 10 minutes at room temperature. Absorbance at 600 nm is then measured for both the reaction wells and the stopped wells. Inhibition can be calculated using the following formulas: Inhibitor (or control) activity=(Inhibitor stopped wells absorbance at 600 nm-inhibitor reaction wells absorbance at 600 nm); Initial activity=control enzyme absorbance at 600 nm; Percent Inhibition=[(Initial activity/Inhibitor Activity)*100]−100.

Peroxisome Proliferator-Activated Receptor Gamma (PPAR-γ) Activity: Changes in the activity of PPAR-γ due to each of the active ingredients, any one of the combination of ingredients, or compositions having said combinations disclosed in the specification can be measured. PPAR-γ is a receptor critical for the production of sebum. As one non-limiting example, the activity of PPAR-γ can be determined using a bioassay that analyzes the ability of a test compound or composition to inhibit binding of a ligand. Briefly, fluorescent small-molecule pan-PPAR ligand, FLUORMONE™ Pan-PPAR Green, available from Life Technologies (PV4894), can be used to determine if test compounds or compositions are able to inhibit binding of the ligand to PPAR-γ. The samples wells include PPAR-γ and fluorescent ligand and either: test compound or composition (test); a reference inhibitor, rosiglitazone (positive control); or no test compound (negative control). The wells are incubated for a set period of time to allow the ligand opportunity to bind the PPAR-γ. The fluorescence polarization of each sample well can then be measured and compared to the negative control well to determine the percentage of inhibition by the test compound or composition.

Cytokine array: Human epidermal keratinocytes are cultured to 70-80% confluency. The media in the plate is aspirated and 0.025% trypsin/EDTA is added. When the cells became rounded, the culture dish is gently tapped to release the cells. The trypsin/EDTA containing cells are removed from the culture dish and neutralized. Cells are centrifuged for 5 min. at 180×g to form a pellet of cells. The supernatant is aspirated. The resulting pellet is resuspended in EPILIFE™ media (Cascade Biologics). The cells are seeded in 6-well plates at approximately 10-20% confluency. After the cells became approximately 80% confluent, the media is aspirated and 1.0 ml of EPILIFE™, along with phorbol 13-Myristate 12-acetate (“PMA”) (a known inducer of inflammation) and the test composition dilutions are added to two replicate wells (i.e., 1.0% (100 μl of 100×stock) and 0.1% (10 μl of 100×stock) test compositions are diluted into a final volume of 1 ml EPILIFE™ Growth Medium). The media is gently swirled to ensure adequate mixing. In addition, 1.0 ml of EPILIFE™ is added to the control wells, with and without additional PMA. The plates are then incubated at 37±1° C. and 5.0±1% CO₂ for approximately 5 hours after dosing. Following this 5-hour incubation, all media is collected in conical tubes and frozen at −70° C.

For analysis, a 16-pad hybridization chamber is attached to 16-pad FAST slides arrayed in triplicate with 16 anti-cytokine antibodies plus experimental controls (WHATMAN BIOSCIENCES), and the slides are placed into a FASTFrame (4 slides per frame) for processing. Arrays are blocked for 15 min. at room temperature using 70 ml S&S PROTEIN ARRAY BLOCKING BUFFER (WHATMAN SCHLEICHER AND SCHEULL). Blocking buffer is removed and 70 ml of each supernatant sample is added to each array. Arrays are incubated for 3 hours at room temperature with gentle agitation. Arrays are washed 3 times with TBS-T. Arrays are treated with 70 ml of an antibody cocktail, containing one biotinylated antibody corresponding to each of the arrayed capture antibodies. Arrays are incubated for 1 hour at room temperature with gentle agitation. Arrays are washed 3 times with TBS-T. Arrays are incubated with 70 ml of a solution containing streptavidin-Cy5 conjugate for 1 hour at room temperature with gentle agitation. Arrays are washed 3 times with TBS-T, quickly rinsed in de-ionized water, and dried.

Slides can be imaged in a PERKIN-ELMER SCANARRAY 4000 confocal fluorescent imaging system. Array images can be saved and analyzed using IMAGING RESEARCH ARRAYVISION software. Briefly, spot intensities are determined by subtracting background signal. Spot replicates from each sample condition can be averaged and then compared to the appropriate controls.

Endothelial Tube Formation: Endothelial tube formation is involved in angiogenesis and micro-vessel capillary formation. Capillary formation and angiogenesis may contribute to redness and rosacea of the skin. The ability for endothelial cells to form tubes in the presence or absence of test extracts and compounds may be determined using a capillary tubule disruption assay with pre-formed primary human umbilical vein endothelial cells (HUVEC) in a cell culture system.

Briefly, HUVECs are cultured in vitro on Extracellular Matrix, which stimulates the attachment and tubular morphogenesis of endothelial cells to form capillary-like lumen structures. These in vitro formed capillary tubules are similar to human blood vessel capillaries in many aspects. The capillary tube assay is based on this phenomenon and is used for evaluation of potential vasculature targeting agents.

HUVEC cultures are grown in a 5% CO₂ 37° C. cell incubator. The full growth medium for HUVECs is Endothelial Cell Basal Medium (EBM) supplemented with 2% fetal bovine serum (FBS), 12 μg/ml bovine brain extract, 1 μg/ml hydrocortisone, and 1 μg/ml GA-1000 (gentamicin-amphothericin). HUVEC cultures between passage 3 and 8 may be used for all assay experiments.

HUVECs are pre-labeled with fluorescent agent Calcein AM and seeded in Extracellular Matrix coated 96-well culture plate with their full growth medium. After about four hours of the morphogenesis process, the endothelial capillary tubes should be formed. Then, test agent in designed doses in 50 μl volume is applied into the formed capillary tubule cultures as treatment conditions. The no-treatment controls can be added with vehicle of test agents. SUTENT®, a FDA approved anti-angiogenic drug one concentration can be included as assay performance control. After about six hours of treatment, the endothelial tubule morphology in each well is examined by microscopy, imaged, and the capillary disrupting activities under treatment conditions can be quantitatively analyzed. Each test conditions can be conducted in duplicate wells, including controls.

The above specification and examples provide a complete description of the structure and use of illustrative embodiments. Although certain aspects have been described above with a certain degree of particularity, or with reference to one or more individual aspects, those skilled in the art could make numerous alterations to the disclosed aspects without departing from the scope of this disclosure. As such, the various illustrative aspects of the methods and systems are not intended to be limited to the particular forms disclosed. Rather, they include all modifications and alternatives falling within the scope of the claims, and aspects other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one aspect or may relate to several aspects.

The claims are not intended to include, and should not be interpreted to include, means plus- or step-plus-function limitations, unless such a limitation is explicitly recited in a given claim using the phrase(s) “means for” or “step for,” respectively.

1. A method of improving the stability of a retinoid in a retinoid-containing composition, the method comprising adding a buffer to the retinoid-containing composition in an amount that is sufficient to maintain a pH of the composition in a pH range of from 5 to 8, wherein the cosmetic composition is an oil-in-water emulsion.

2. The method of claim 1, wherein the retinoid is selected from retinol, retinal, retinoic acid, branched or unbranched C1-C20 esters of retinoic acid, etretinate, acitretin, adapalene, bexarotene, tazarotene, and any combination thereof.

3. The method of claim 1, wherein the retinoid is added in an amount ranging from 0.01 wt. % to 5 wt. % relative to a weight of the composition.

4. The method of claim 1, wherein the retinoid is added in an amount ranging from 1 wt. % to 3 wt. % relative to a weight of the composition.

5. The method of claim 1, wherein the buffer is selected from the group consisting of tris(hydroxymethyl)aminomethane (TRIS), citrate phosphate, 2-(N-morpholino)ethanesulfonic acid (MES), 3-(N-morpholino)propanesulfonic acid (MOPS), 4-(N-morpholino)butanesulfonic acid (MOBS), bis(2-hydroxyethyl)amino-tris(hydroxymethyl)methane (BIS-TRIS), N-(2-acetamido) iminodiacetic acid (ADA), N-(2-acetamido)-2-aminoethanesulfonic acid (ACES), 1,4-piperazinediethanesulfonic acid (PIPES), 3-morpholino-2-hydroxypropane sulfonic acid (MOPSO), 1,3-bis[tris(hydroxymethyl)methylamino]propane (BIS-TRIS propane), N,N-bis-(2-hydroxyethyl)-2-aminoethanesulfonic acid (BES), 2-[(2-hydroxy-1,1-bis-(hydroxymethyl)ethyl)amino]ethanesulfonic acid (TES), 4-(2-hydroxyethyl) piperazine-1-ethanesulfonic acid (HEPES), 2-hydroxy-3-[tris(hydroxymethyl)methylamino]-1-propanesulfonic acid (TAPSO), 2-amino-2-(hydroxymethyl)-1,3-propanediol (trizma), piperazine-N,N′-bis(2-hydroxypropanesulfonic acid) (POPSO), 4-(2-hydroxyethyl)-1-piperazinepropanesulfonic acid (EPPS), Diglycine, N,N-bis(2-hydroxyethyl)glycine (BICINE), N-(2-hydroxyethyl)piperazine-N′-(4-butanesulfonic acid) (HEPBS), 2-amino-2-methyl-1,3-propanediol, and N-[tris(hydroxymethyl)methyl]-3-aminopropanesulfonic acid (TAPS).

6. The method of claim 1, wherein the buffer is added in an amount ranging from 0.01 wt. % to 5 wt. % relative to the weight of the composition.

7. The method of claim 1, further comprising adding at least one antioxidant to the retinoid-containing composition.

8. The method of claim 7, wherein the at least one antioxidant is at least one water-soluble antioxidant, at least one oil-soluble antioxidant, or a combination thereof.

9. The method of claim 8, wherein the at least one water-soluble antioxidant is selected from the group consisting of citric acid, a citrate salt, ascorbic acid, an ascorbate salt, and a combination thereof.

10. The method of claim 8, wherein the at least one oil-soluble antioxidant is selected from the group consisting of tocopherol, a tocopherol ester, and a combination thereof.

11. The method of claim 9, wherein the at least one water-soluble antioxidant is added in an amount ranging from 0.01 wt. % to 5 wt. % relative to the weight of the composition.

12. The method of claim 10, wherein the at least one oil-soluble antioxidant is added in an amount ranging from 0.01 wt. % to 5 wt. % relative to the weight of the composition.

13. The method of claim 1, further comprising adding at least one chelating agent to the retinoid-containing composition.

14. The method of claim 13, wherein the at least one chelating agent is selected from disodium EDTA, tetrasodium EDTA, citric acid, glucono δ-lactone, sodium phytate, and any combination thereof.

15. The method of claim 14, wherein the at least one chelating agent is added in an amount ranging from 0.01 wt. % to 5 wt. % relative to the weight of the composition.

16. The method of claim 1, wherein the method increases retinoid stability such that at least 90% by weight of an original amount of the retinoid remains after storage for 12 weeks at 40° C.

17. A cosmetic composition comprising at least one retinoid and a buffer, wherein the buffer is present in an amount that is sufficient to maintain a pH of the composition in a pH range of from 5 to 8, and wherein the cosmetic composition is in the form of an oil-in-water emulsion.

18. The cosmetic composition of claim 17, wherein the composition exhibits enhanced retinoid stability such that at least 90% by weight of an original amount of the retinoid in the cosmetic composition remains after storage for 12 weeks at 40° C.

19. The cosmetic composition of claim 17, further comprising at least one of an emulsifier, an emulsion thickening agent, an emulsion stabilizer, a surfactant, a texture enhancer, an emollient, a humectant, a preservative, a pH-adjusting agent, and a cleansing agent.

20. A method for extending the effectiveness of a retinoid-containing composition, comprising adding a buffer to the retinoid-containing composition in an amount that is sufficient to maintain a pH of the composition in a pH range of from 5 to 8, wherein the wherein the cosmetic composition is an oil-in-water emulsion.
